SEQUENCE LISTING (14 sequences)

SEQ ID NO: 1 - DNA, 43 bp, Artificial Sequence
  Synthetic
ggccgccccgggcgataaatatagtacctaaggtaaattacac

SEQ ID NO: 2 - DNA, 91 bp, Artificial Sequence
  Synthetic
aaattataataaactgcaggccttagtgatggtgatggtgatgcccgggagttggcggggccttcagtggcctttctccccatccatcatc

SEQ ID NO: 3 - DNA, 24 bp, Artificial Sequence
  Synthetic
cataaccatttcgcgtaagattca

SEQ ID NO: 4 - DNA, 23 bp, Artificial Sequence
  Synthetic
ttgaccattaaagtttgctgcta

SEQ ID NO: 5 - DNA, 24 bp, Artificial Sequence
  Synthetic
actaattgttctacccagcctaaa

SEQ ID NO: 6 - DNA, 22 bp, Artificial Sequence
  Synthetic
tgttccattatcttcgtgctca

SEQ ID NO: 7 - DNA, 24 bp, Artificial Sequence
  Synthetic
aagaaatgaaaaatgggatgaggt

SEQ ID NO: 8 - DNA, 23 bp, Artificial Sequence
  Synthetic
aacggttagaagattttgatgct

SEQ ID NO: 9 - DNA, 24 bp, Artificial Sequence
  Synthetic
tggactttacaggatactcaggaa

SEQ ID NO: 10 - DNA, 20 bp, Artificial Sequence
  Synthetic
gcatcacaggcaggcgtaga

SEQ ID NO: 11 - DNA, 38 bp, Artificial Sequence
  Synthetic
ccggcagtaactctggcaatggccctcatccacgctgg

SEQ ID NO: 12 - DNA, 38 bp, Artificial Sequence
  Synthetic
ccagcgtggatgagggccattgccagagttactgccgg

SEQ ID NO: 13 - DNA, 44 bp, Artificial Sequence
  Synthetic
ccggcagtaactctggcaatggccctcatccaagctggtcaccg

SEQ ID NO: 14 - DNA, 38 bp, Artificial Sequence
  Synthetic
cggtgaccagcttggatgagggccattgccagagttac

(19) United States
(12) Patent Application Publication
(10) Pub. No.: US 2016/0051644 A1
(43) Pub. Date: Feb. 25, 2016

(54) Botulinum Chimera Compositions for Axonal Regenerative Therapy During Spinal Cord Injury

(71) Applicant: Bal Ram Singh, (US)

(72) Inventors: Bal Ram Singh, North Dartmouth, MA (US); Nagarajan Thirunavukkarasu, New Bedford, MA (US)

(21) Appl. No.: 14/761,373

(22) PCT Filed: Jan. 16, 2014
(86) PCT No.: PCT/US14/11796

§ 371 Date: Jul. 16, 2015

Related U.S. Application Data
(60) Provisional application No. 61/753,178, filed on Jan. 16, 2013.

(51) Int. Cl. A61K 38/48; C12N 9/52; C12N 9/10; A61K 38/45; A61K 35/74

(52) U.S. Cl. CPC A61K 38/4893; A61K 38/45; A61K 35/74; C12N 9/1077; C12N 9/52; C12Y 304/24069; C12Y 204/02

(57) ABSTRACT

The present invention relates to the isolation of polypeptides derived from the Clostridium botulinum neurotoxin and the use thereof as treatment for neuronal injury such as spinal cord injury. Botulinum neurotoxin binds to neural cells and are translocated into the cytosol and therefore is useful as a target specific therapeutic delivery system. Non-toxic botulinum toxin may be created by light chain mutagenesis and/or removal of the endopeptidase domain.

### FIELD OF THE INVENTION

The present invention relates to compositions and methods to use Clostridium botulinum neurotoxin (BoNT) for the treatment of spinal cord injury, to regenerate injured axons. For example, the compositions described herein provide a Clostridium botulinum neurotoxin heavy chain (BoNT(HC)). Alternatively, the compositions described herein provide a chimeric protein comprising a BoNT(HC). In particular, the chimeric protein may further comprise a C3E-exoenzyme. Such compositions may be administered to patients having spinal cord injury, wherein the BoNT(HC) targets the injured spinal cord.

### BACKGROUND OF THE INVENTION

The genus Clostridium is comprised of gram-positive, anaerobic, spore-forming bacilli. The natural habitat of these organisms is the environment and the intestinal tracts of humans and other animals. Indeed, Clostridia bacteria are ubiquitous; they are commonly found in soil, dust, sewage, marine sediments, decaying vegetation, and mud. See e.g., P.H.A. Sneath et al., “Clostridium,” Bergey's Manual of Systematic Bacteriology, Vol. 2, pp. 1141-1200, Williams & Wilkins (1986). Despite the identification of approximately 100 species of Clostridium, only a small number have been recognized as etiologic agents of medical and veterinary importance. Nonetheless, these species are associated with very serious diseases, including botulism, tetanus, anaerobic cellulitis, gas gangrene, bacteremia, pseudomembranous colitis, and clostridial gastroenteritis.

Clostridium botulinum toxin is commonly used in several formulations (for e.g., Botox™, Dysport™, Xeomin™, Myoblock™) to treat neuromuscular disorders associated with excessive muscle contraction such as strabismus, blepharospasm, hemifacial spasm and cervical dystonia, various types of pain and in cosmetics to remove facial wrinkles due to its ability to block neurotransmitter release and mediators of pain. Shauna et al., “Botulinum toxin in neurological diseases” Saudi Arab. J. Rehab. 10:111-117 (2004). BoNTs block exocytosis in central neurons with similar potencies and durations matching those of motor nerve terminals and in their ability to alter glutamate, noradrenaline, dopamine, and glycine transmission, along with electrophysiological properties, in vitro and in vivo animal studies. Costantin et al., “Antiepileptic effects of botulinum neurotoxin E” J. Neurosci. 25:1943-1951 (2005). BoNTs are also used as a therapeutic agent in individuals with generalized spasticity secondary to SCI, improving pain, by injecting into muscle groups and in combination with rehabilitative therapy.

Besides the utility of BoNTs for several therapeutic applications, BoNTs or its non-toxic derivatives of BoNTs would be highly useful as a drug carrier. Full-length derivatives of BoNTs (˜150 kDa) and the heavy chain (HC) derivatives of clostridial neurotoxins have been demonstrated for its potential to deliver drug candidates (Singh et al., 2010). Given its potential to deliver drugs, the transport or trafficking characterstics of BoNTs can also be exploited to deliver drugs to central nervous system by injecting them through minimally-invasive intra muscular route of administration. Several clinical data demonstrate bilateral antinociceptive effects of BoNT/A implicating its actions on CNS following unilateral, peripheral administration of BoNT/A toxin. Some of the observed intraspinal effects of toxin had been suggested by its potential to ascend retrogradely along the peripheral branch of nociceptive neurons and then penetrating the spinal cord through anterograde transport and transcytosis across synapses. Their ability to undergo dose-dependent, microtubule based retrograde transport, and neuronal transcytosis in both CNS, and motoneurons of peripheral nervous system without being degraded. Caleo and Schiavo, 2009. Central effects of tetanus and botulinum neurotoxins. Toxicon. 54, 593-599; and Caleo et al., 2009. A reappraisal of the central effects of Botulinum Neurotoxin TypeA: By what mechanism? J. Neurochem. 109, 15-24. Using the visual pathway as a model system, BoNT/A was experimentally shown to undergo anterograde axonal transport and transcytosis across synapses, providing explanation on the mechanisms of BoNT/A direct actions in pain management. Restani et al., 2011. Evidence for anterograde transport and transcytosis of botulinum neurotoxin A. (BoNT/A). J Neurosci. 31, 15650-9. Such trafficking characteristics suggest the possibility of using BoNT/A fragments as drug delivery vehicles targeting the central nervous system, and thus non-toxic BoNT/A form new class of carriers for delivering therapeutic agents into the central nervous system (Restani et al., 2012. Botulinum neurotoxin A impairs neurotransmission following retrograde transynaptic transport. Traffic. 13, 1083-9).

Spinal Cord Injury (SCI) is a physically disabling condition which can lead to paraplegia or quadriplegia affecting significant impact on quality of life, life expectancy and economic burden, with considerable costs associated with primary care and loss of income. An estimate (year-2009) from. National Spinal Cord Injury Statistical Center indicates that the present survivors in United States of SCI are in the range of 229,000 to 306,000 persons, with approximately 12,000 new cases each year. Studies report that quadriplegics ranked recovery of arm and hand function as a priority, whereas paraplegics rated recovery of sexual function as most important, when measured against recovery of bladder/bowel function, and eradicating autonomic dysreflexia, improving walking movements and trunk stability, regaining normal sensation and eliminating chronic pain. Thuret et al., “Therapeutic interventions for spinal cord injury” Nat. Rev. Neurosci. 7:628-643 (2006). Besides this, other important priorities are recovery of cardiovascular performance and skeletomuscular properties, and reducing spasticity. Therapeutic options for SCI are very limited and current therapeutic measures for Spinal Cord Injury (SCI) involve combinatorial approaches tailoring various rehabilitative, cellular and molecular therapies.

What is needed in the art is a drug product that could be potentially used by topical application or intramuscular application to prophylactically or therapeutically protect or promote regeneration of severed axons during spinal cord injury.

### SUMMARY OF THE INVENTION

The present invention relates to compositions and methods to use Clostridium botulinum neurotoxin (BoNT) for the treatment of spinal cord injury. For example, the compositions described herein provide a Clostridium botulinum neurotoxin heavy chain (BoNT(HC)). Alternatively, the compositions described herein provide a chimeric protein comprising a BoNT(HC). In particular, the chimeric protein may further comprise a C3E-exoenzyme. Such compositions may be administered to patients having spinal cord injury, wherein the BoNT(HC) targets the injured spinal cord.

In one embodiment, the present invention contemplates a composition comprising a chimeric protein comprising a C3E-exoenzyme protein and a non-toxic neurotoxin heavy chain. In one embodiment, the neurotoxin heavy chain comprises a Clostridium botulinum neurotoxin heavy chain. In one embodiment, the chimeric protein further comprises neurotoxin light chain. In one embodiment the neurotoxin light chain lacks an endopeptidase region. In one embodiment the neurotoxin light chain lacks an endopeptidase activity. In one embodiment, the neurotoxin light chain comprises at least one mutation. In one embodiment, the neurotoxin comprises at least two mutations. In one embodiment, the at least one mutation is selected from at least one of the group including, but not limited to, H223M, H227Q, E224A or E262A. In one embodiment, the C3E-exoenzyme protein is a Clostridium botulinum C3E-exoenzyme protein. In one embodiment, the C3E protein and the heavy chain are linked by a disulfide bridge. In one embodiment, the C3E protein is attached to the neurotoxin light chain. In one embodiment, the C3E protein is linked to the receptor-binding domain of Clostridium botulinum neurotoxin heavy chain. In one embodiment, C3E is linked to the translocation domain of the Clostridium botulinum neurotoxin heavy chain.

In one embodiment, the present invention contemplates a method, comprising: a) providing; i) a patient suspected of having a nerve tissue injury; ii) a composition comprising a chimeric protein comprising a C3E protein and a non-toxic neurotoxin heavy chain; b) administering the composition to the patient wherein said nerve injury is at least partially regenerated. In one embodiment, the nerve tissue injury comprises a spinal cord injury. In one embodiment, the administering includes, but is not limited to topical, intramuscular, intraspinal or intrathecal. In one embodiment, the neurotoxin heavy chain comprises a Clostridium botulinum neurotoxin heavy chain. In one embodiment, the chimeric protein further comprises neurotoxin light chain. In one embodiment, the neurotoxin light chain comprises at least one mutation. In one embodiment the neurotoxin light chain lacks an endopeptidase region. In one embodiment the neurotoxin light chain lacks an endopeptidase activity. In one embodiment, the neurotoxin comprises at least two mutations. In one embodiment, the at least one mutation is selected from at least one of the group including, but not limited to, H223M, H227Q, E224A or E262A. In one embodiment, the C3E-exoenzyme protein is a Clostridium botulinum C3E-exoenzyme protein. In one embodiment, the C3E protein and the heavy chain are linked by a disulfide bridge. In one embodiment, the C3E protein is attached to the neurotoxin light chain. In one embodiment, the C3E protein is linked to the receptor-binding domain of Clostridium botulinum neurotoxin heavy chain. In one embodiment, C3E is linked to the translocation domain of the Clostridium botulinum neurotoxin heavy chain.

In one embodiment, the present invention contemplates an isolated nucleic acid sequence encoding a chimeric protein comprising a C3E-exoenzyme protein and a non-toxic neurotoxin heavy chain. In one embodiment, the neurotoxin heavy chain comprises a Clostridium botulinum neurotoxin heavy chain. In one embodiment, the chimeric protein further comprises neurotoxin light chain. In one embodiment the neurotoxin light chain lacks an endopeptidase region. In one embodiment the neurotoxin light chain lacks an endopeptidase activity. In one embodiment, the neurotoxin light chain comprises at least one mutation. In one embodiment, the neurotoxin comprises at least two mutations. In one embodiment, the at least one mutation is selected from at least one of the group including, but not limited to, H223M, H227Q, E224A or E262A. In one embodiment, the C3E-exoenzyme protein is a Clostridium botulinum C3E-exoenzyme protein. In one embodiment, the C3E protein and the heavy chain are linked by a disulfide bridge. In one embodiment, the C3E protein is attached to the neurotoxin light chain. In one embodiment, the C3E protein is linked to the receptor-binding domain of Clostridium botulinum neurotoxin heavy chain. In one embodiment, C3E is linked to the translocation domain of the Clostridium botulinum neurotoxin heavy chain.

In one embodiment, the nucleic acid sequence is ligated to a vector. In one embodiment, the vector comprises a pBN3 vector. In one embodiment, the vector is transfected into a host cell. In one embodiment, the host cell comprises an E. coli cell.

In one embodiment, the present invention contemplates a drug delivery system comprising a composition comprising a C3E-exoenzyme, a non-toxic neurotoxin heavy chain and a carrier. In one embodiment, the heavy chain comprises a sulfhydryl group. In one embodiment, the heavy chain is attached to the C3E-exoenzyme. In one embodiment, the C3E-exoenzyme comprises a Clostridium botulinum C3E-exoenzyme. In one embodiment, the neurotoxin heavy chain comprises a Clostridium botulinum neurotoxin heavy chain. In one embodiment, the chimeric protein further comprises neurotoxin light chain. In one embodiment the neurotoxin light chain lacks an endopeptidase region. In one embodiment, the neurotoxin light chain comprises at least one mutation. In one embodiment, the neurotoxin comprises at least two mutations. In one embodiment, the at least one mutation is selected from at least one of the group including, but not limited to, H223M, H227Q, E224A or E262A. In one embodiment, the drug delivery system further comprises a medical device capable of administering the composition to an injured tissue. In one embodiment, the injured tissue comprises neuronal tissue. In one embodiment, the neuronal tissue comprises spinal cord tissue. In one embodiment, the spinal cord tissue comprises spinal cord axons. In one embodiment, the carrier comprises a liposome. In one embodiment, the carrier comprises a microparticle. In one embodiment, the medical device includes, but is not limited to, a catheter, a sprayer, and/or a tube. In one embodiment, the chimeric protein further comprises a drug. In one embodiment, the drug includes, but is not limited to, anti-inflammatory, corticosteroid, antithrombotic, antibiotic, antibacterial, antifungal, antiviral, analgesic, dextran, and anesthetic drugs. In one embodiment, the drug includes, but is not limited to, peptides, proteins, polypeptides and/or fragments thereof. In one embodiment, the drug includes, but is not limited to, nucleic acids, polynucleic acids and/or fragments thereof. In one embodiment, the nucleic acid comprises silencing RNA (siRNA). In one embodiment, the nucleic acid comprises interfering RNA (RNAi). In one embodiment, the polynucleic acid comprises a sense nucleic acid sequence. In one embodiment, the polynucleic acid comprises an antisense nucleic acid sequence.

### DEFINITIONS

As used herein, the term “overproducing” is used in reference to the production of clostridial toxin polypeptides in a host cell and indicates that the host cell is producing more of the clostridial toxin by virtue of the introduction of nucleic acid sequences encoding said clostridial toxin polypeptide than would be expressed by said host cell absent the introduction of said nucleic acid sequences. To allow ease of purification of toxin polypeptides produced in a host cell it is preferred that the host cell express or overproduce said toxin polypeptide at a level greater than 1 mg/liter of host cell culture.

As used herein, the term “fusion protein” or “a chimeric protein” refers to any protein containing a protein of interest (i.e., for example, a neurotoxin A or B and fragments thereof, a C3E-exoenzyme, a Clostridium botulinum toxin heavy chain etc.) joined to an exogenous protein and/or fragment thereof. The fusion partner may enhance solubility of an expressed recombinant protein from a host cell and/or target a therapeutic protein within a patient of interest. If desired, the fusion protein may be removed from the protein of interest (i.e., toxin protein or fragments thereof) prior to immunization by a variety of enzymatic or chemical means known to the art.

As used herein the term “non-toxic protein” or “non-toxin protein” refers to that portion of a fusion/chimeric protein which comprises a protein or a protein sequence which is not derived from a bacterial toxin protein, or lacks a region that has toxic activity (e.g., an endopeptidase region), or comprises mutations in a region that eliminates toxic activity (e.g., BoNT point mutations including, but not limited to, H223M, H227Q, E224A or E262A). For example, a non-toxic protein may be a non-toxic Clostridium botulinum protein subtype A (DR BoNT/A)

The term “protein of interest” as used herein refers to the protein whose expression is desired within the fusion protein. In a fusion protein the protein of interest will be joined or fused with another protein or protein domain, the fusion partner, to allow for enhanced stability of the protein of interest and/or ease of purification of the fusion protein.

As used herein, the term “maltose binding protein” refers to the maltose binding protein of E. coli. A portion of the maltose binding protein may be added to a protein of interest to generate a fusion protein; a portion of the maltose binding protein may merely enhance the solubility of the resulting fusion protein when expressed in a bacterial host. On the other hand, a portion of the maltose binding protein may allow affinity purification of the fusion protein on an amylose resin.

As used herein, the term “poly-histidine tract” when used in reference to a fusion protein refers to the presence of two to ten histidine residues at either the amino- or carboxy-terminus of a protein of interest. A poly-histidine tract of six to ten residues is preferred. The poly-histidine tract is also defined functionally as being a number of consecutive histidine residues added to the protein of interest which allows the affinity purification of the resulting fusion protein on a nickel-chelate column.

As used herein, the term “purified” or “to purify” refers to the removal of contaminants from a sample. For example, antitoxins are purified by removal of contaminating non-immunoglobulin proteins; they are also purified by the removal of immunoglobulin that does not bind toxin. The removal of non-immunoglobulin proteins and/or the removal of immunoglobulins that do not bind toxin results in an increase in the percent of toxin-reactive immunoglobulins in the sample. In another example, recombinant toxin polypeptides are expressed in bacterial host cells and the toxin polypeptides are purified by the removal of host cell proteins; the percent of recombinant toxin polypeptides is thereby increased in the sample. Additionally, the recombinant toxin polypeptides are purified by the removal of host cell components such as lipopolysaccharide (e.g., endotoxin).

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed from a recombinant DNA molecule.

The term “native protein” as used herein refers to a protein which is isolated from a natural source as opposed to the production of a protein by recombinant means.

As used herein the term “portion” when in reference to a protein (as in “a portion of a given protein”) refers to fragments of that protein. The fragments may range in size from four amino acid residues to the entire amino acid sequence minus one amino acid.

As used herein “soluble” when in reference to a protein produced by recombinant DNA technology in a host cell is a protein which exists in solution in the cytoplasm of the host cell; if the protein contains a signal sequence the soluble protein is exported to the periplasmic space in bacteria hosts and is secreted into the culture medium in eukaryotic cells capable of secretion or by bacterial host possessing the appropriate genes (i.e., the kil gene). In contrast, an insoluble protein is one which exists in denatured form inside cytoplasmic granules (called an inclusion bodies) in the host cell. High level expression (i.e., greater than 10-20 mg recombinant protein/liter of bacterial culture) of recombinant proteins often results in the expressed protein being found in inclusion bodies in the bacterial host cells. A soluble protein is a protein which is not found in an inclusion body inside the host cell or is found both in the cytoplasm and in inclusion bodies and in this case the protein may be present at high or low levels in the cytoplasm.

A distinction is drawn between a soluble protein (i.e., a protein which when expressed in a host cell is produced in a soluble form) and a “solubilized” protein. An insoluble recombinant protein found inside an inclusion body may be solubilized (i.e., rendered into a soluble form) by treating purified inclusion bodies with denaturants such as guanidine hydrochloride, urea or sodium dodecyl sulfate (SDS). These denaturants must then be removed from the solubilized protein preparation to allow the recovered protein to renature (refold). Not all proteins will refold into an active conformation after solubilization in a denaturant and removal of the denaturant. Many proteins precipitate upon removal of the denaturant. SDS may be used to solubilize inclusion bodies and will maintain the proteins in solution at low concentration. However, dialysis will not always remove all of the SDS (SDS can form micelles which do not dialyze out); therefore, SDS-solubilized inclusion body protein is soluble but not refolded.

A distinction is drawn between proteins which are soluble (i.e., dissolved) in a solution devoid of significant amounts of ionic detergents (e.g., SDS) or denaturants (e.g., urea, guanidine hydrochloride) and proteins which exist as a suspension of insoluble protein molecules dispersed within the solution. A soluble protein will not be removed from a solution containing the protein by centrifugation using conditions sufficient to remove bacteria present in a liquid medium (i.e., centrifugation at 5,000×g for 4-5 minutes). For example, to test whether two proteins, protein A and protein B, are soluble in solution, the two proteins are placed into a solution selected from the group consisting of PBS-NaCl (PBS containing 0.5 M NaCl), PBS-NaCl containing 0.2% Tween 20, PBS, PBS containing 0.2% Tween 20, PBS-C (PBS containing 2 mM CaCl2), PBS-C containing either 0.1 or 0.5% Tween 20, PBS-C containing either 0.1 or 0.5% NP-40, PBS-C containing either 0.1 or 0.5% Triton X-100, PBS-C containing 0.1% sodium deoxycholate. The mixture containing proteins A and B is then centrifuged at 5000×g for 5 minutes. The supernatant and pellet formed by centrifugation are then assayed for the presence of protein A and B. If protein A is found in the supernatant and not in the pellet except for minor amounts (i.e., less than 10%) as a result of trapping, protein is said to be soluble in the solution tested. If the majority of protein B is found in the pellet (i.e., greater than 90%), then protein B is said to exist as a suspension in the solution tested.

As used herein, the term “therapeutic amount” refers to that amount of antitoxin required to neutralize the pathologic effects of one or more clostridial toxins in a subject.

The term “pyrogen” as used herein refers to a fever-producing substance. Pyrogens may be endogenous to the host (e.g., prostaglandins) or may be exogenous compounds (e.g., bacterial endo- and exotoxins, nonbacterial compounds such as antigens and certain steroid compounds, etc.). The presence of pyrogen in a pharmaceutical solution may be detected using the U.S. Pharmacopeia (USP) rabbit fever test (United States Pharmacopeia, Vol. XXII (1990) United States Pharmacopeial Convention, Rockville, Md., p. 151).

The term “endotoxin” as used herein refers to the high molecular weight complexes associated with the outer membrane of gram-negative bacteria. Unpurified endotoxin contains lipids, proteins and carbohydrates. Highly purified endotoxin does not contain protein and is referred to as lipopolysaccharide (LPS). Because unpurified endotoxin is of concern in the production of pharmaceutical compounds (e.g., proteins produced in E. coli using recombinant DNA technology), the term endotoxin as used herein refers to unpurified endotoxin. Bacterial endotoxin is a well known pyrogen.

As used herein, the term “endotoxin-free” when used in reference to a composition to be administered parenterally (with the exception of intrathecal administration) to a host means that the dose to be delivered contains less than 5 EU/kg body weight FDA Guidelines for Parenteral Drugs (December 1987). Assuming a weight of 70 kg for an adult human, the dose must contain less than 350 EU to meet FDA Guidelines for parenteral administration. Endotoxin levels are measured herein using the Limulus Amebocyte Lysate (LAL) test (Limulus Amebocyte Lysate Pyrochrome™, Associates of Cape Cod, Inc. Woods Hole, Mass.). To measure endotoxin levels in preparations of recombinant proteins, 0.5 ml of a solution comprising 0.5 mg of purified recombinant protein in 50 mM NaPO4, pH 7.0, 0.3M NaCl and 10% glycerol is used in the LAL assay according to the manufacturer's instructions for the endpoint chromogenic without diazo-coupling method. Compositions containing greater than or equal less than 60 endotoxin units (EU)/mg of purified recombinant protein are herein defined as “substantially endotoxin-free.” Typically, administration of bacterial toxins or toxoids to adult humans for the purpose of vaccination involves doses of about 10-500 μg protein/dose. Therefore, administration of 10-500 μg of a purified recombinant protein to a 70 kg human, wherein said purified recombinant protein preparation contains 60 EU/mg protein, results in the introduction of only 0.6 to 30 EU (i.e., 0.2 to 8.6% of the maximum allowable endotoxin burden per parenteral dose).

The LAL test is accepted by the U.S. FDA as a means of detecting bacterial endotoxins (21 C.F.R. §§660.100-105). Studies have shown that the LAL test is equivalent or superior to the USP rabbit pyrogen test for the detection of endotoxin and thus the LAL test can be used as a surrogate for pyrogenicity studies in animals F. C. Perason, Pyrogens: endotoxins, LAL testing and clepyrogenation, Marcel Dekker, New York (1985), pp. 150-155. The FDA Bureau of Biologics accepts the LAL assay in place of the USP rabbit pyrogen test so long as the LAL assay utilized is shown to be as sensitive as, or more sensitive as the rabbit test Fed. Reg., 38, 26130 (1980).

The term “monovalent” when used in reference to a clostridial vaccine refers to a vaccine which is capable of provoking an immune response in a host animal directed against a single type of clostridial toxin. For example, if immunization of a host with C. botulinum type A toxin vaccine induces antibodies in the immunized host which protect against a challenge with type A toxin but not against challenge with type B, C, D, E, or F toxins, then the type A vaccine is said to be monovalent. In contrast, a “multivalent” vaccine provokes an immune response in a host animal directed against several (i.e., more than one) clostridial toxins. For example, if immunization of a host with a vaccine comprising C. botulinum type A and B toxins induces the production of antibodies which protect the host against a challenge with both type A and B toxin, the vaccine is said to be multivalent (in particular, this hypothetical vaccine is bivalent).

As used herein the term “immunogenically-effective amount” refers to that amount of an immunogen required to invoke the production of protective levels of antibodies in a host upon vaccination.

The term “protective level”, when used in reference to the level of antibodies induced upon immunization of the host with an immunogen which comprises a bacterial toxin, means a level of circulating antibodies sufficient to protect the host from challenge with a lethal dose of the toxin.

As used herein the terms “protein” and “polypeptide” refer to compounds comprising amino acids joined via peptide bonds and are used interchangeably.

The terms “toxin” and “neurotoxin” when used in reference to toxins produced by members (i.e., species and strains) of the genus Clostridium are used interchangeably and refer to the proteins which are poisonous to nerve tissue.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents exemplary Western Blot data showing anti-BoNT/A antibody binding to either DR BoNT/A protein (Lane 1) or native BoNT/A protein (Lane 3). Kaleidoscope prestained marker standard was used for visualization (Lane 2).

FIG. 2 presents exemplary SDS-PAGE electrophoresis data showing the approximate molecular weights of DR BoNT/A (Lane 2) and native BoNT/A (Lane 3).

FIG. 3 presents exemplary circular dichroism data showing absorption maxima at 208 and 222 nm for both DR BoNT/A protein and native BoNT/A protein.

FIG. 4 presents exemplary trypsinization fragmentation patterns of native BoNT/A protein under reducing and non-reducing conditions.

FIG. 5 presents exemplary trypsinization fragmentation patterns of DR BoNT/A protein under reducing and non-reducing conditions.

FIG. 6 presents exemplary isoelectric focusing data showing approximate pI's for either DR BoNT/A (Lane 2) and native BoNT/A (Lane 3).

FIG. 7 presents exemplary data showing the integration of BoNT/A heavy chain (HC) into a pBN3 vector double mutant light chain (LC).

FIG. 8 presents exemplary endopeptidase data showing the activity in the L chain and native BoNT/A protein, but not the double mutant L chain, DR BoNT/A protein, or recombinant H chain.

FIG. 9 presents exemplary data showing cell membrane binding of DR BoNT/A.

FIG. 9A: Blue-fluorescent Hoechst 33342 shows nucleus.

FIG. 9B: Green-fluorescent FITC shows the binding of DR BoNT/A to SH-SY5Y cells.

FIG. 9C: Red-fluorescent Alexa Fluor 594 shows the plasma membrane.

FIG. 9D: merged images showing DR BoNT/A bound to the plasma membrane.

FIG. 10 presents SDS PAGE gel electrophoresis of one embodiment of a triple mutant BoNT/A; H223M/E224A/E262A BoNT/A (DR BoNT/A-T).

FIG. 11 presents SDS PAGE gel electrophoreses of one embodiment of a quadruple mutant BoNT/A; H223M/E224A/H227Q/E262A BoNT/A (DR BoNT/A-Q)

FIG. 12 presents a schematic overview of one embodiment for testing recombinant BoNT-C3E chimera in axonal regenerative therapy.

FIG. 12A: Proposed mechanism of BoNT/A utility in hypercholinergic and pain disorders; The endopeptidase domain or light chain (LC/A, in blue) is linked through disulfide bridge to the translocation domain (TD, in green) which essential to deliver the cargo across endosomal vesicles, and receptor binding domain (RD in red), the neurotrophic determinant.

FIG. 12B: Proposed mechanism of BoNT based C3E delivery system as drug for axon regeneration. A DR BoNT-C3E chimera comprises a C3E fragment fusion with a non-toxic, catalytically deactivated version of BoNT/A. A BoNT(HC)/A-C3E chimera comprises a C3E fragment fusion with a BoNT/(HC)/A attached by a disulfide linkage that may be cleaved for intracellular C3E delivery.

FIG. 13 presents an illustration of injured spinal cord showing damaged myelinated axons & myelin debris. Enlarged view A: Myelin debris and collapsed growth cone.

FIG. 14A presents a schematic diagram of BoNT/A domains. Specific amino acid residues are designated in single letter code and position number.

FIG. 14B presents a three-dimensional protein ribbon illustration of the BoNT/A crystal structure. Yellow and red: HC comprising a receptor binding domain (RBD); Green: translocation domain (TD); Blue: light chain (LC) comprising the endopeptidase domain.

FIG. 15 present exemplary data showing the relative binding and internalization of DR BoNT/A-Alexa488 in human neuroblastoma, SH-SY5Y and rhabdomyosarcoma cells

FIG. 16 presents an illustration of one embodiment of a BoNT drug delivery vehicle (DDV) construct with Oregon green dye.

FIG. 17 presents exemplary data showing a series of confocal images demonstrating cellular binding and/or intracellular compartmentalization of DDV drug delivery.

FIG. 18 presents exemplary data showing an in vivo mouse model toxicity assay for BoNT, BoNT(HC)/A and DR BoNT/A.

FIG. 19 presents a schematic overview of comparative advantages for a DR BoNT/A based C3E delivery system to regenerate axons after SCI to other existing therapies.

FIG. 20 presents a schematic diagram (not in exact scale) of exemplary recombinant constructs containing different domains with a C-terminal 6× His-Tag. Yellow and red: HC/A domain comprising a receptor binding domain (RBD) and HC1+HC2 sub-domains; Green: HC/A domain comprising a translocation domain (TD); Blue: LC/A domain comprising a 25 amino acid stretch containing a protease nicking site and cysteine residue. Pink: a fused C3E exoenzyme.

FIG. 20A: BoNT/A (150 kDa).

FIG. 20B: BoNT/A-C3E, C3E chimera with RBD and TD (123 kDa);

FIG. 20C: BoNT(HC)/A with RBD and TD (100 kDa);

FIG. 20D: C3E (23 kDa) with an engineered cysteine residue (—SH).

### DETAILED DESCRIPTION OF THE INVENTION

The present invention relates to compositions and methods to use Clostridium botulinum neurotoxin for the treatment of spinal cord injury. For example, the compositions described herein provide a Clostridium botulinum neurotoxin heavy chain (BoNT(HC)). Alternatively, the compositions described herein provide a chimeric protein comprising a BoNT(HC). In particular, the chimeric protein may further comprise a C3E-exoenzyme. Such compositions may be administered to patients having spinal cord injury, wherein the BoNT(HC) targets the injured spinal cord.

Different strains of Clostridium botulinum each produce antigenically distinct toxin designated by the letters A-G. Serotype A toxin has been implicated in 26% of the cases of food botulism; types B, E and F have also been implicated in a smaller percentage of the food botulism cases. H. Sugiyama, Microbiol. Rev. 44:419 (1980). Wound botulism has been reportedly caused by only types A or B toxins H. Sugiyama, supra. Nearly all cases of infant botulism have been caused by bacteria producing either type A or type B toxin. (Exceptionally, one New Mexico case was caused by Clostridium botulinum producing type F toxin and another by Clostridium botulinum producing a type B-type F hybrid.) S. Arnon, Epidemiol. Rev. 3:45 (1981). Type C toxin affects waterfowl, cattle, horses and mink. Type D toxin affects cattle, and type E toxin affects both humans and birds.

### I. Spinal Cord Injuries

Spinal Cord Injury (SCI) is a physically disabling condition with devastating social emotional psychological and life style effects, mostly victimizing the young healthy people in their ripe productive years. Worldwide, an estimated 2.5 million people live with spinal cord injury (SCI), with more than 130,000 new injuries reported each year. An estimate (year-2009) from National Spinal Cord Injury Statistical Center indicates that the present survivors in United States of SCI are in the range of 229,000 to 306,000 persons, with approximately 12,000 new cases each year. Studies report that quadriplegics ranked recovery of arm and hand function as a priority, whereas paraplegics rated recovery of sexual function as most important, when measured against recovery of bladder/bowel function, and eradicating autonomic dysreflexia, improving walking movements and trunk stability, regaining normal sensation and eliminating chronic pain. Thuret et al., “Therapeutic interventions for spinal cord injury” Nat. Rev. Neurosci. 7:628-643 (2006). Other identified recovery priorities were cardiovascular performance, skeletomuscular properties, and reducing spasticity. Currently available therapeutic options for SCI are very limited, affecting significant impact on quality of life, life expectancy and economic burden, with considerable costs associated with primary care and loss of income.

A. Axonal Injury

Spinal cord injury (SCI) can lead to paraplegia or quadriplegia and current treatment involves combinatorial approaches tailoring various rehabilitative, cellular and molecular therapies. Current molecular therapies for SCI are generally aimed to modulate neuronal survival, neurite outgrowth, enhance synaptic plasticity and/or neurotransmission. An alternative molecular therapeutic approach promotes axonal regeneration after SCI by targeting molecules that are intrinsic to neurons. Current development in promoting axonal regeneration after SCI has been achieved by blocking an ability of RhoA activation using C3 Exoenzyme (C3E), a 24 kDa ADP-ribosyltransferase, isolated from Clostridium botulinum. Although it is not necessary to understand the mechanism of an invention, it is believed that RhoA, in its active GTP-bound form, rigidifies the actin cytoskeleton, thereby inhibiting axonal elongation and mediating growth cone collapse. Although C3E is believed to regenerate injured axons, an effective delivery of C3E at the axonal lesion sites remains a major challenge in developing clinical applications for this enzyme as an effective therapy (i.e., for example, C3E does not have a cell binding domain).

Various research groups are currently exploring molecular targets to manage and treat SCI, but there are only limited opportunities when one considers suitable methods and strategies to deliver therapeutic drugs like C3E. Consequently, there is strong need to develop an efficient, safe alternative non-viral based drug delivery system to augment neurotherapeutics, in general. In one embodiment, the present invention contemplates alternatives to non-viral drug delivery systems for the treatment of SCI. In one embodiment, the non-viral drug delivery system comprises a non-toxic Botulinum Neurotoxin (BoNT) based C3E delivery system. Although it is not necessary to understand the mechanism of an invention, it is believed that a BoNT/C3E delivery system blocks RhoA signaling for axonal regenerative therapy of SCI.

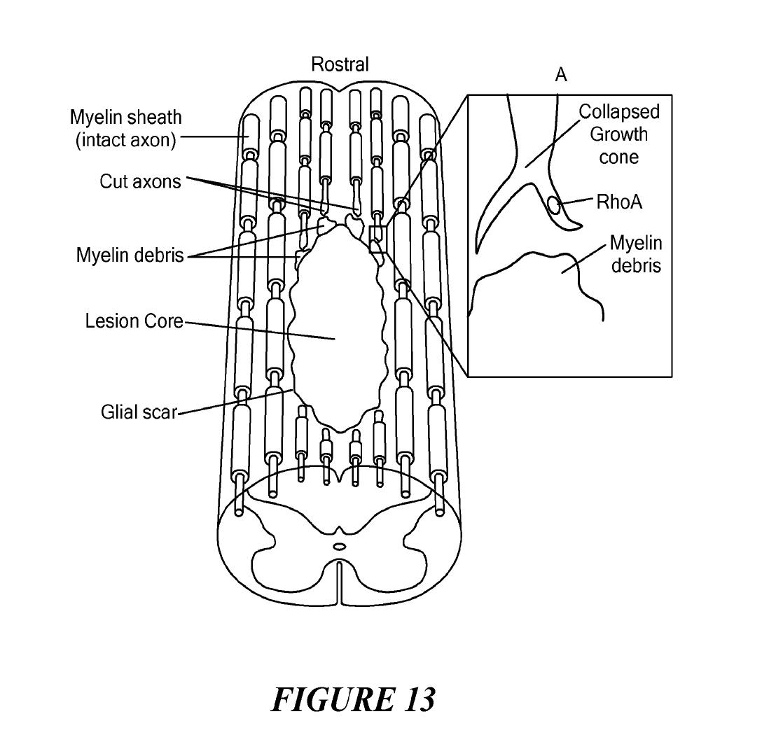

In SCI, injured axons in the adult central nervous system (CNS) do not grow spontaneously to restore connectivity. Cellular events subsequent to the acute phase of SCI typically include, but are not limited to, post-traumatic necrosis of severed neurons and non-neuronal cells, along with lesser degree of apoptotic death of neurons and glia in the vicinity of the injury, followed by reactive vascular changes which cause secondary damage to the spinal cord. Kakulas, B. A., “Neuropathology:the foundation for new treatments in spinal cord injury” Spinal Cord 42:549-563 (2004). SCI symptomology may also be accompanied by wallerian degeneration of an injured neuron which is characterized by structural destruction of axolemma, a dismantled axonal cytoskeleton at the distal end and myelin sheath breakage. These reactive glial events eventually lead to a white scar-like tissue, consisting of two main zones, the lesion-core populated by meningeal fibroblasts, vascular endothelial cells, and frequently by oligodendrocyte precursors (OPCs) and the lesion-surrounding area including, but not limited to, reactive astrocytes, OPCs, and microglia. See, FIG. 13.

Most CNS axons stop regenerating within an area surrounding a lesion, while a few may penetrate the surrounding area. Eventually, a regenerating axon may reach the lesion core boundary and invariably stop regenerating with the development of swollen dystrophic ends, with the lesion-core being the absolute regeneration barrier. Hermanns et al., “The collagenous lesion scar—an obstacle for axonal regeneration in brain and spinal cord injury” Restor. Neural. Neurosci. 19:139-148 (2001). Thus, inhibitory molecules mediating the physical glial scar barriers of axonal regeneration include, but are not limited to, myelin-derived molecules, Nogo, myelin-associated glycoprotein (MAG), OMgp, and ephrinB3, as well as the astrocyte-scar-enriched chondroitin sulfate proteoglycans (CSPGs). Harel et al., “Can regenerating axons recapitulate developmental guidance during recovery from spinal cord injury?” Nat. Rev. Neurosci. 7:603-616 (2006); and Wang et al., “Ibuprofen Enhances Recovery from Spinal Cord Injury by Limiting Tissue Loss and Stimulating Axonal Growth” J. Neurotrauma 26:81-95 (2009).

Interestingly, many myelin-derived inhibitors signal through a common receptor complex (e.g., NgR, p75/TROY and Lingo complex) to activate RhoA, as are the CSPGs through a less understood signaling mechanism. Thus, RhoA may be involved in several different signaling mechanisms of different inhibitory cues relevant to axonal injuries. Dergham et al., “Rho signaling pathway targeted to promote spinal cord repair” J. Neurosci. 22:6570-6577 (2002); Fournier et al., “Rho kinase inhibition enhances axonal regeneration in the injured CNS” J. Neurosci. 23:1416-1423 (2003); Jin et al., “Rac1 mediates collapsin-1-induced growth cone collapse” J. Neurosci. 17:6256-6263 (1997); Lehmann et al., “Inactivation of Rho signaling pathway promotes CNS axon regeneration” J. Neurosci. 19:7537-7547 (1999); and Niederost et al., “Nogo-A and myelin-associated glycoprotein mediate neurite growth inhibition by antagonistic regulation of RhoA and Rac1” J. Neurosci. 22:10368-10376 (2002).

RhoA is believed to be an intracellular GTP-binding protein that is involved in regulating actin filament polymerization and organization. RhoA in its active GTP-bound form rigidifies the actin cytoskeleton, thereby causing growth cone collapse, inhibiting axonal elongation and cell body rounding. Winton et al., “Characterization of new cell-permeable C3-like proteins that inactivate Rho and stimulate neurite outgrowth on inhibitory substrates” J. Biol. Chem. 277:32820-2829 (2002); Niederost et al., “Nogo-A and myelin-associated glycoprotein mediate neurite growth inhibition by antagonistic regulation of RhoA and Rac1” J. Neurosci. 22:10368-10376 (2002); and Schweigreiter et al., “Versican V2 and the central inhibitory domain of Nogo-A inhibit neurite growth via p75NTR/NgR-independent pathways that converge at RhoA” Mol. Cell. Neurosci. 27:163-174 (2004). RhoA is believed to be abnormally activated in injured axons distal from an SCI lesion site and considered as one of the factors accelerating Wallerian degeneration. Madura et al., “Activation of Rho in the injured axons following spinal cord injury” EMBO Rep. 5:412-417 (2004); and Yamagishi et al., “Wallerian degeneration involves Rho/Rho-kinase signaling” J. Biol. Chem. 280:20384-20388 (2005).

In several attempts to promote recovery from spinal cord injury by reducing RhoA activity, it was observed that irreversible inhibition of RhoA effector domain by ADP-ribosylation at asparagine-41 by C3 exoenzyme (C3E) from Clostridium botulinum directly promotes axonal growth in primary cortical neurons in vitro may be achieved independent of its GTP-bound state. Dergham et al., “Rho signaling pathway targeted to promote spinal cord repair” J. Neurosci. 22:6570-6577 (2002). It should be noted that C3E produced by Clostridium botulinum apparently has no role as a bacterial virulence factor and has an extremely low toxicity in mice (i.e., for example, about 400 μg/kg I.P.) Rappuoli, R., and Montecucco, C. (Eds) 1997, Guidebook to protein toxins and their use in cell biology. A Sambrook and Tooze Publication at Oxford University Press.

In adult SCI mice models, RhoA inhibition was reported to show histological long-distance regeneration of anterogradely labeled corticospinal axons, and was correlated to behavioral recovery of locomotion followed by forelimb-hindlimb coordination. Dergham et al., “Rho signaling pathway targeted to promote spinal cord repair” J Neurosci. 22:6570-6577 (2002). There is also evidence that inactivation of RhoA by C3E is effective in stimulating axon regeneration across glial scars, both in vitro and in vivo within a therapeutic window. Furthermore, application of C3E on neuronal cell bodies promotes regeneration of distal axons, suggesting C3E is able to access its target at the nerve terminal. Bertrand et al., “Application of Rho antagonist to neuronal cell bodies promotes neurite growth in compartmented cultures and regeneration of retinal ganglion cell axons in the optic nerve of adult rats”J. Neurosci. 25:1113-1121 (2005).

RhoA inhibitors, including but not limited to, C3E, also have neuroprotective effects in addition to promoting axon regeneration. Dubreuil et al., “Rho activation patterns after spinal cord injury and the role of activated Rho in apoptosis in the central nervous system” J. Cell Biol. 162:233-243 (2003). RhoA inhibitors have also been shown histologically to reduce the lesion area/volume by preventing apoptotic cell-death of neurons and/or glial cells thus establishing additional benefits for acute SCI therapy. Lord-Fontaine et al., “Local Inhibition of Rho Signaling by Cell-Permeable Recombinant Protein BA-210 Prevents Secondary Damage and Promotes Functional Recovery following Acute Spinal Cord Injury” J. Neurotrauma 25:1309-1322 (2008); and Fournier et al., “Rho kinase inhibition enhances axonal regeneration in the injured CNS” J. Neurosci. 23:1416-1423 (2003). Notably, RhoA associated inhibition is a potential therapeutic strategy, as demonstrated in animal models of stroke and Alzheimer's disease. Kubo et al., “The therapeutic effects of Rho-ROCK inhibitors on CNS disorders” Ther. Clin. Risk. Manag. 4:605-615 (2008).

B. Conventional Regeneration Treatments

Various rehabilitative, cellular and molecular therapies have been tested in SCI animal models. Current therapeutic efforts are combinatorial in approach depending on the nature, degree, site of injury and may also depend according to the stage examined being acute, subacute or chronic. These cellular and molecular therapeutics aim to reverse neuropathology. Some cellular therapies may replace dead cells (for example, with new neurons or myelinating cells) and/or create a favorable environment for axon regeneration. These are achievable by transplantation of peripheral nerve, Schwami cells from peripheral nerve, olfactory nervous system cells, embryonic CNS tissue, stem/progenitor cells or through, ex-vivo engineered stem/progenitor cells. Thuret et al., “Therapeutic interventions for spinal cord injury” Nat. Rev. Neurosci. 7:628-643 (2006).

Other molecular therapies for SCI aim to modulate neuronal survival by neuroprotection, neurite outgrowth, or to enhance synaptic plasticity and neurotransmission. The effectiveness of these approaches have been tested on lesioned spinal cord by different routes and delivery methods, using, for example, brain-derived neurotrophic factor (BDNF), basic fibroblast growth factor, glial cell-derived neurotrophic factor (GDNF) nerve growth factor (NGF) and neurotrophin 3 (NT3), NT4 and NT5.

Unfortunately, clinical trials using systemic delivery of growth factors for various disorders have failed either as a result of lack of efficacy or unacceptable side effects, or both, and it is also considered that delayed delivery of growth factors might be less effective than acute delivery because axons of chronically injured neurons may lack appropriate growth factor receptors. Due to such complexities, targeting molecules intrinsic to neurons that could promote axonal regeneration has an uninvestigated potential. For example, elevating cyclic AMP (cAMP) did not regenerate SCI in mammals although induced axonal sprouting in in vitro cell had previously been observed. Another testing strategy was to promote axonal regeneration with target molecules that are intrinsic to injured neurons. For example, those molecules are reported to modulate GTPases through Rho and Rae and effect neuronal cytoskeleton signal transduction. Schweigreiter et al., “Versican V2 and the central inhibitory domain of Nogo-A inhibit neurite growth via p75NTR/NgR-independent pathways that converge at RhoA” Mol. Cell. Neurosci. 27:163-174 (2004); and Lord-Fontaine et al., “Local Inhibition of Rho Signaling by Cell-Permeable Recombinant Protein BA-210 Prevents Secondary Damage and Promotes Functional Recovery following Acute Spinal Cord Injury” J. Neurotrauma. 25:1309-1322 (2008). However, none of these reports identify suitable methods and strategies to deliver therapeutics that will be effective, safe, minimally invasive and compatible to treat the associated neuropathology of SCI.

A. C3E Exoenzyme SCI Therapy

C3E (C3E-Exoenzyme) produced by Clostridium botulinum is known to regenerate injured axons during spinal cord injury (SCI). C3E is believed to act by irreversibly inhibiting RhoA effector domain by ADP-ribosylation at asparagine-41 to directly promote axonal growth in primary cortical neurons in vitro. For example, in adult mice models of SCI, RhoA inhibition showed histological long-distance regeneration of anterogradely labeled corticospinal axons, behavioral recovery of locomotion followed by forelimb-hind limb coordination and thus establishing C3E as a therapeutic drug. Dergham et al., “Rho signaling pathway targeted to promote spinal cord repair” J. Neurosci. 22:6570-6577 (2002). A cell permeable version of C3E (BA-205) has been shown to prevent secondary damage following acute spinal cord injury, and also shown to promote functional recovery” Fontaine et al., J. Neurotrauma 25:1309-1322 (2008).

C3E has been used for treating axonal regeneration of SCI with varying degrees of success. For example, in a study treating small neuronal scars, axon regeneration was not observed after intrathecal C3E application, possibly because C3E lacks any transport sequence and is not able to enter damaged nerves in the spinal cord. Fournier et al., “Rho kinase inhibition enhances axonal regeneration in the injured CNS” J. Neurosci. 23:1416-1423 (2003). C3E therapy has significant limitations because the C3E protein does not have a natural cell binding component to allow efficient entry mechanisms into cells other than pinocytosis or related mechanisms. Han et al., “Crystal structure and novel recognition motif of Rho ADP-ribosylating C3 exoenzyme from Clostridium botulinum: structural insights for recognition specificity and catalysis” J. Mol. Biol. 305:95-107 (2001).

Various disruptive methods have been reported in an effort to facilitate the entry of C3E into cells. For example, C3E was microinjected into individual fibroblast cells or cellular entry was aided by triturating or scrape-loading techniques in neuronal cells. Jin et al., “Rac1 mediates collapsin-1-induced growth cone collapse” J. Neurosci. 17:6256-6263 (1997); and Lehmann et al., “Inactivation of Rho signaling pathway promotes CNS axon regeneration” J. Neurosci. 19:7537-7547 (1999), respectively.

Cell-permeable versions of C3E have also been tested for delivery. A fusion between C3E and the B subunit of diphtheria toxin (DT) that binds to cell surface receptors and is internalized by an endocytic mechanism has been attempted. But since rodents lack DT receptors, the constructs cannot be used to conduct animal studies and also are not useful for neuronal targeting. Several C3E fusions with Tat protein of human immunodeficiency virus, Antp peptide of Antennapedia homeodomain, and praline or arginine rich peptides have been tested. Winton et al., “Characterization of new cell-permeable C3-like proteins that inactivate Rho and stimulate neurite outgrowth on inhibitory substrates” J. Biol. Chem. 277:32820-32829 (2002).

Recently, a proline rich C3E fusogenic derivative, C3-05 (or BA-205) was selected for human clinical trials using a fibrin sealant formulation with an endpoint of SCI axonal growth restoration following topical application to the lesion site. Lord-Fontaine et al., “Local Inhibition of Rho Signaling by Cell-Permeable Recombinant Protein BA-210 Prevents Secondary Damage and Promotes Functional Recovery following Acute Spinal Cord Injury” J. Neurotrauma 25:1309-1322 (2008). A Tat-C3 membrane permeating fusion was recently developed that promoted neurite outgrowth, and was further encapsulated in biocompatible polymer poly (D, L-lactide-co-glycolide) for sustainable controlled release of the protein, but its efficiency of drug delivery in relevant SCI models is not known. Tan et al., “Development of a cell transducible RhoA inhibitor TAT-C3 transferase and its encapsulation in biocompatible microspheres to promote survival and enhance regeneration of severed neurons” Pharma. Res. 24:2297-2308 (2007).

Although the therapeutic potential of C3E is well documented, there are significant challenges to overcome before C3E becomes a safe and effective clinical therapeutic:

a) C3E is Limited to Non-Specific Cell Delivery

Neuronal cells are believed to be the primary target cell in which RhoA signaling inhibition would be expected to augment SCI axonal regenerative therapy. Currently, C3E is administered using non-specific cell entry mechanism by employing cell-permeable C3E variants. Since the inhibition of RhoA in normal cells are not involved in SCI pathology, a non-specific C3E administration is likely to exert toxicity. This toxicity may result from many pathways for which RhoA signaling is known to be involved including, but not limited to, general cell physiology, the regulation of cell shape change, cytokinesis, cell adhesion, and/or cell migration. Lu et al., “Signaling through Rho GTPase pathway as viable drug target” Curr Med Chem. 16(11):1355-1365 (2009). Furthermore, tolerance shown by cell-permeable C3E variants in SCI animal models could be quite different to that of human SCI, thus making neuron-selective drug delivery systems a superior and advantageous method of administration. Indeed, it is increasingly recognized to incorporate neuron-targeting ligands for better transfection efficiencies, and to reduce toxicity due to widespread entry in off-target cells, while developing non-viral mediated neuronal delivery system. Bergen et al., “Analysis of the intracellular barriers encountered by nonviral gene carriers in a model of spatially controlled delivery to neurons” J Gene Med. 10(2):187-197 (2008).

b) C3E is Limited to a Topical Route of Delivery

Preferred techniques to administer molecular therapies in SCI animal models include, but are not limited to, intracerebroventricular injection, intrathecal injection, intraspinal injection, continuous infusion, and/or insertion of a carrier saturated with the molecule of interest. Nonetheless, current clinical trials with C3E using fibrin sealant formulation are limited to a topical administration directly to the lesion site. Lord-Fontaine et al., “Local Inhibition of Rho Signaling by Cell-Permeable Recombinant Protein BA-210 Prevents Secondary Damage and Promotes Functional Recovery following Acute Spinal Cord Injury” J. Neurotrauma 25:1309-1322 (2008). Although minimally invasive and/or traumatic routes of administration of C3E (e.g., intraspinal, intrathecal etc.) are preferable because of limited motor neuron availability, C3E delivery with non-viral carriers have not yet been reported. Among less invasive routes, intramuscular administration using retrograde transporting carriers for use in SCI therapy have been discussed. Bergen et al., “Analysis of the intracellular barriers encountered by nonviral gene carriers in a model of spatially controlled delivery to neurons” J Gene Med. 10(2):187-197 (2008).

c) Limitations of Other Viral and/or Non-Viral Carriers

Although several molecular targets are currently being explored to manage and treat SCI, there are only limited opportunities when one considers suitable methods and strategies to deliver therapeutic drugs. There is a great necessity to develop efficient, safe alternative non-viral based delivery systems to augment neurotherapeutics in general, since neuronal cells are highly differentiated cells with somal domains, axonal domains, and dendritic domains wherein each domain has distinct membrane compositions.

Several viral and non-viral carrier based gene delivery methods have been described in literature as feasible strategies for CNS neuronal delivery. However, only a few have been successful in neurological disease or injury animal models. For example, in vivo gene therapy has been tested in SCI animal models using viruses including, but not limited to, herpes simplex virus (HSV), adenovirus, Adeno-Associated Virus (AAV), lentivirus and/or Moloney leukaemia virus. However, injection of HSV into mammalian tissue elicited a local immune response. Additionally, AAV clinical trials were delayed due to production of neutralizing antibodies in humans possibly by previous exposure and often encounter safety issues. Bergen et al., “Analysis of the intracellular barriers encountered by nonviral gene carriers in a model of spatially controlled delivery to neurons” J Gene Med. 10(2):187-197 (2008).

Other currently existing non-viral therapeutic carriers for neuronal gene delivery suffer from low transfection efficiencies and/or have toxicity issues as compared to that of neurotropic viral deliveries. It is believed that the low efficiency of these non-viral carriers is due to non-specific binding to most cell surfaces. Consequently, the internalization of these non-viral carriers vary depending on the neuronal cell type, size, charge, and surface composition, the delivery site, and the specific vehicle formulation, etc. Without doubt, delivering sufficient quantities of a therapeutic under these conditions that would have an effect with restricted distribution to the targeted site is difficult.

Controlling the expression levels and/or durations of a therapeutic transgene also has limitations resulting in suboptimal dosing and/or overdosing. For example, polyethylenimine (PEI)/DNA polyplexes, and cationic lipid-based lipoplex-mediated delivery to neurons were shown to aggregate in biological fluids. Berry et al., “Gene therapy for central nervous system repair” Curr. Opin. Mol. Ther. 3:338-349 (2001). Additionally, PEI exhibits cellular toxicity and reduced uptake in neuron-like cells as compared to that of undifferentiated cells. Polyplexes and lipoplexes, when delivered to neurite terminals, undergo internalization in the vesicles that are part of endo-lysosomal pathway. Bergen et al., “Analysis of the intracellular barriers encountered by nonviral gene carriers in a model of spatially controlled delivery to neurons” J Gene Med. 10(2):187-197 (2008). Polyplexes and lipoplexes inability to escape from the acidification of endosomal compartments in neurons partially explains their poor transfection efficiencies compared to viruses. Suk et al., “Quantifying the intracellular transport of viral and nonviral gene vectors in primary neurons” Exp. Biol. Med. 232:461-469 (2007).

### II. Botulinum Neurotoxins

Botulinum neurotoxins (BoNTs) are a group of extremely potent toxins, which are produced by various strains of Clostridium botulinum, and in some cases by C. butirycum and C. barati. Clostridial neurotoxins comprise seven serotypes (A-G) of botulinum neurotoxins, each produced by Clostridium botulinum as a 150 kDa single peptide chain. Sollner et al., Nature 362, 318-324 (1993). Besides being the most poisonous toxin known to humankind, BoNTs are also FDA-approved to treat different neuromuscular disorders and various types of pain. BoNTs are neurotoxins with an ability to block neurotransmitter release in both peripheral and central neurons. BoNT is known to provide effective treatment for several neuromuscular related disorders and for treating various types of pain.

Clostridial neurotoxins include, but are not limited to, botulinum neurotoxin (BoNT) and/or Tetanus neurotoxin (TeNT). Both BoNT and TeNT belong to an “A-B” toxin group because both toxins comprise an enzymatically active component (A) and cell binding component (B). BoNT is post-translationally proteolyzed to form a dichain in which the heavy chain (HC) and light chain (LC) are linked through a disulfide bond. Montecucco et al., Q. Rev. Biophys 28:423-472 (1995). BoNTs possess an enzymatically active 50 kDa light chain (LC) and a 100 kDa heavy chain (HC). HC is composed of two 50 kDa domains, with the N-terminal portion involved in translocation, and C-terminal portion involved in cellular binding. On the other hand, the LC comprises an enzymatic activity region (i.e., for example, an endopeptidase region). Montecucco et al., “Structure and function of tetanus and botulinum neurotoxins” Quart. Rev. Biophys. 28:423-472 (1995); Singh et al., “Intimate details of the most poisonous poison” Nat. Struct. Biol. 7:617-619 (2000); and Singh, B. R., “Botulinum neurotoxin structure, engineering, and novel cellular trafficking and targeting” Neurotoxicity Res. 9:73-92 (2006). As part of their physiological mode of action, BoNTs are generally known to bind with presynaptic cholinergic nerve cells at the peripheral neuromuscular junctions and block acetylcholine release causing flaccid paralysis by receptor mediated endocytosis.

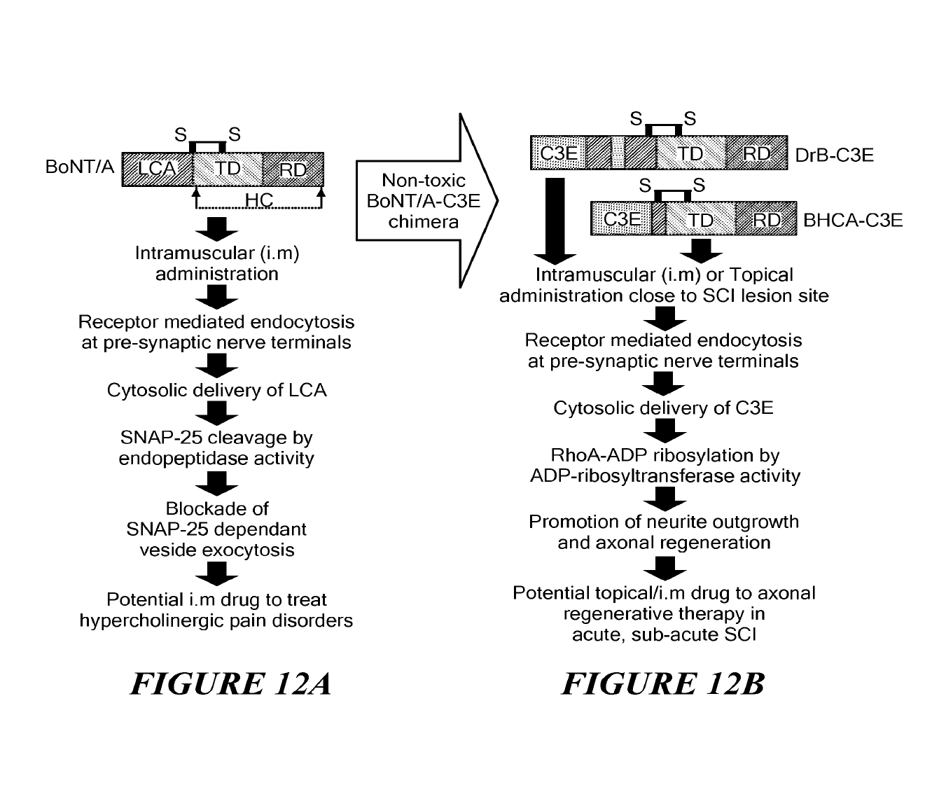

The HC is organized into at least two distinct trimodular structural domains that perform different functional features; i) a C-terminal receptor binding domain (RD, red) that binds to the peripheral neuromuscular junction presynaptic nerve terminal synaptic vesicle protein SV2C isoforms and/or the ganglioside lipid acceptors; ii) a translocation domain (TD, green), which facilitates light chain (LC/A, green)) translocation across the cell endosomal membrane, thereby providing access to cytosolic SNARE targets. See FIG. 12A. Although it is not necessary to understand the mechanism of an invention it is believed that LC/A cleaves SNAP-25, a SNARE protein involved in exocytosis, thereby blocking acetylcholine release. It is further believed that LC/A may also inhibit neurotransmitter release involved in pain transmission, which are reported therapeutics for the treatment of hypercholinergic disorders and various types of pain. BoNT/A is also a prospective therapeutic for several CNS disorders, since they could also block release of neurotransmitter from the synaptosome of brain, spinal cords, and brain primary nerve cell cultures by entering during SV recycling like in peripheral terminals. Interestingly, it is believed that BoNT/A injections are accompanied by formation of motor axon sprouts and the RD domain of BoNT/A possess neuritogenic potentials, which could be useful in SCI therapy.

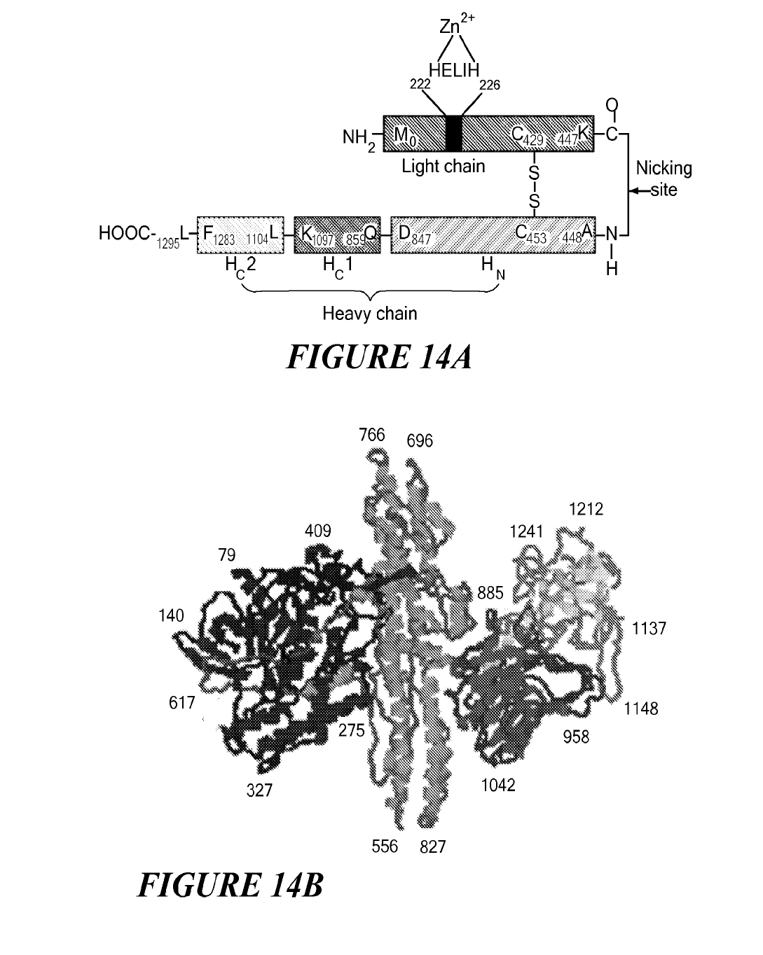

One process of BoNT cell intoxication can be described as follows: i) binding; ii) internalization; iii) membrane translocation; and iv) inhibition of neurotransmitter release. Grumelli et al., “Internalization and mechanism of action of clostridial toxins in neurons” Neurotoxicol. 26:761-767 (2005). BoNT serotype A (BoNT/A) selectively binds to acceptors on the surface of presynaptic membrane through the HC C-terminal Receptor Binding Domain (RBD) and is internalized via receptor mediated endocytosis. A 25 kDa C-terminal sub-domain HC2 of a BoNT/A HC binds to luminal domains of intracellular components including, but not limited to, synaptic vesicle (SV) glycoproteins (i.e., for example, isoforms SV2A, SV2B and SV2C) and/or polysialogangliosides. See, FIGS. 14A and 14B. With gene knockout experiments, it was also demonstrated the SV isoforms also seem to complement BoNT binding and associated toxicity (data not shown).

Upon acidification of endosomes, the N-terminal domain of HC/A forms a pore or transmembrane channel to translocate the BoNT/A light chain (LC/A) into neuronal cytoplasm. The N-terminus of the translocation domain (TD) that wraps the LC/A has been implicated as a regulatory loop for membrane interaction during acidification. Galloux et al., “Membrane interaction of botulinum neurotoxin A-T domain: The belt region is a regulatory loop for membrane interaction” J. Biol. Chem. 283:27668-27676 (2008), and FIG. 14B.

Once endocytosis is complete, a release of the LC/A may spontaneously occur by cleavage of the interchain disulfide bond linking the HC/A and LC/A because of the high reduction potential of the cytosol. Additionally, a single chain BoNT may be cleaved by cellular proteases to separate and deliver (e.g., release) an LC or other therapeutic cargo into the cytosol. The presence of the disulfide bond is also shown to play a role in the translocation process. The BoNT/A LC released in the cytoplasm cleaves SNAP-25 (e.g., Synaptosome Associated Protein of MW 25 kDa), a soluble NSF-attachment-protein receptor (SNARE) protein in the vesicle recycling machinery by its Zinc dependent endopeptidase activity, thus intervening the process of synaptic vesicle docking and fusion (exocytosis) involved in the acetylcholine at the nerve-muscle junctions. LC of other serotypes also blocks neurotransmitter release by endopeptidase activity against several other isoforms of SNARE proteins like VAMP and syntaxin. Singh, B. R., “Botulinum neurotoxin structure, engineering, and novel cellular trafficking and targeting” Neurotoxicity Res. 9:73-92 (2006).

Although it is not necessary to understand the mechanism of an invention, it is believed that LC works as a zinc endopeptidase to cleave specifically one of the three different SNARE proteins essential for synaptic vesicle fusion (Montecucco and Schiavo, G (1993) Trends Biochem. Sci. 18, 324-327; Li and Singh, B. R. (1999) Toxin Rev. 18, 95-112). In one embodiment, BoNT/A and/or BoNT/E cleave SNAP-25. In another embodiment, TeNT and/or BoNT/B, /D, /F and /G cleave cellubrevin. In one embodiment, BoNT/C cleaves syntaxin and SNAP-25. Once a SNARE protein is cleaved, the release of a neurotransmitter (i.e., for example, acetylcholine) is prevented, ultimately leading to the flaccid muscle paralysis (Montecucco and Schiavo, “Structure and function of tetanus and botulinum neurotoxins” Q. Rev. Biophys, 28:423-472 (1995). However, BoNTs, in general, and BoNT/A in particular, is known to bind to a cell membrane, translocate into the intracellular space, cleave SNAP-25, and block release of neurotransmitter synaptic vesicles within brain and spinal cord cells and/or brain primary nerve cell cultures. Habermann E., “¹²⁵I-labeled neurotoxin from Clostridium botulinum A: preparation, binding to synaptosomes and ascent to the spinal cord” Naunyn Schmiedebergs Arch. Pharmacol. 281:47-56 (1974); Bigalke et al., “Tetanus toxin and botulinum A toxin inhibit release and uptake of various transmitters, as studied with particulate preparations from rat brain and spinal cord” Naunyn Schmiedebergs Arch. Pharmacol. 316:244-251 (1981); Sharma et al., “Botulinum toxin in neurological diseases” Saudi Arab. J Rehab. 10:111-117 (2004). Recently, it was also shown that direct injection of BoNT/A in mice brain also has effect on their nerve functions. Luvisetto et al., “Central injection of botulinum neurotoxins: behavioural effects in mice” Behav. Pharmacol. 15:233-240 (2004). These observations suggest that BoNT/A binding and translocation domains will be fully effective in binding and translocation of cargo to the central nervous system, thus the spinal cord nerves, as targeted for drug delivery for SCI.

A botulinum neurotoxin active site is believed to comprise of a HEXXH+E zinc-binding motif (Li et al., (2000) Biochemistry 39:2399-2405). Type A botulinum neurotoxin crystallography has revealed that H223, H227, and E262 of the HEXXH+E motif directly coordinate the zinc, and E224 coordinates a water molecule as the fourth ligand (Lacy et al., (1998) Nat Struct Biol. 5:898-902). The general conformation and active site residues appear conserved in all of the clostridial neurotoxins (Agarwal et al., (2005) Biochemistry 44, 8291-8302).

The BoNTs are typical zinc metalloproteases which have unique conserved zinc binding motif (HEXXH+E) in the active site. Although it is not necessary to understand the mechanism of an invention, it is believed that the zinc may be coordinated by two histidines, a glutamate and a water molecule, presumably playing a role in the catalytic activity. For example, the amino acid residues in BoNT/A active site comprise H²²³-E²²⁴-L²²⁵-I²²⁶-H²²⁷+E²⁶²

### III. Non-Toxic Botulinum Neurotoxin Compositions

In one embodiment, the present invention contemplates compositions and methods directed to a non-toxic recombinant botulinum toxin A (DR BoNT/A). In one embodiment, a DR BoNT/A is created by site-specific mutation (i.e., for example, by point mutations of LC amino acids). In one embodiment, a DR BoNT/A is created by removing an enzymatic activity region (i.e., for example, an LC endopeptidase region).

A. DR BoNT/A Created by Mutagenesis

For therapeutic delivery, some embodiments of the present invention contemplate an inactive BoNT Light Chain (LC). In one embodiment, the inactive BoNT(LC) comprises a catalytically inactive full-length version of BoNT/A. In one embodiment, a BoNT(LC)/A comprises at least one mutated amino acid residue. In one embodiment, the BoNT(LC)/A comprises at least two mutated amino acid residues. infra; and Yang et al., “Expression, purification and comparative characterization of deactivated recombinant botulinum neurotoxin type A” The Botulinum J. 1:219-241 (2009).

Site-directed mutation studies carried out on BoNT/A and/or TeNT LC demonstrate that active site mutations result in either drastically reduced (E224D) or completely abolished (E224Q) endopeptidase activity. (Li et al., (2000) Biochemistry 39:2399-2405; Binz et al., (2002) Biochemistry 41:1717-1723; Rigoni et al., Biochemistry and Biophys, Res. Commun 288, 1231-1237; Rossetto et al., (2001) Toxicon 39:1150-1159). Although it is not necessary to understand the mechanism of an invention, it is believed that this loss of activity is due to an interference with the hydrolysis step and may not be due to a change in binding affinity of either SNAP-25 or the Zn²⁺ ligand to the enzyme (Li et al., (2000) Biochemistry 39:2399-2405). Further, crystal structure and mutagenesis studies have shown that there may be additional distal amino acids residues act as a secondary coordination sites of zinc. Specifically, secondary coordination sites may stabilize zinc binding and substrate specificity. Sharma et al., Biochemistry 43:4791-4798 (2004).

A basic understanding of BoNT's endopeptidase activity and receptor binding activity are currently known, however, the translocation process is not well understood. Truncated recombinant LC or HC have been utilized mainly due to the poor availability and extreme toxicity of native holo-toxin. Consequently, the present invention contemplates a non-toxic form of the holo-toxin to be utilized for further research and vaccine development. In one embodiment, the non-toxic holo-toxin is created in a recombinant protein expression system. Experiments conducted during the course of development of embodiments of the present invention resulted in a plasmid harboring the full length BoNT/A gene with two active site E224A/E262A mutations in a His-tagged construction. For example, a full length protein was expressed in E. coli as a soluble form, and the biochemical properties were characterized in comparison with the native holo-toxin. The detoxified recombinant BoNT/A (DR BoNT/A) showed no lethality to mice at a 100,000 to 1 million mouse LD₅₀ dose. DR BoNT/A characterizations for molecular size and amino acid sequence match native BoNT/A. DR BoNT/A, however, lacks endopeptidase activity against SNAP-25.

1. Western Blot Analysis

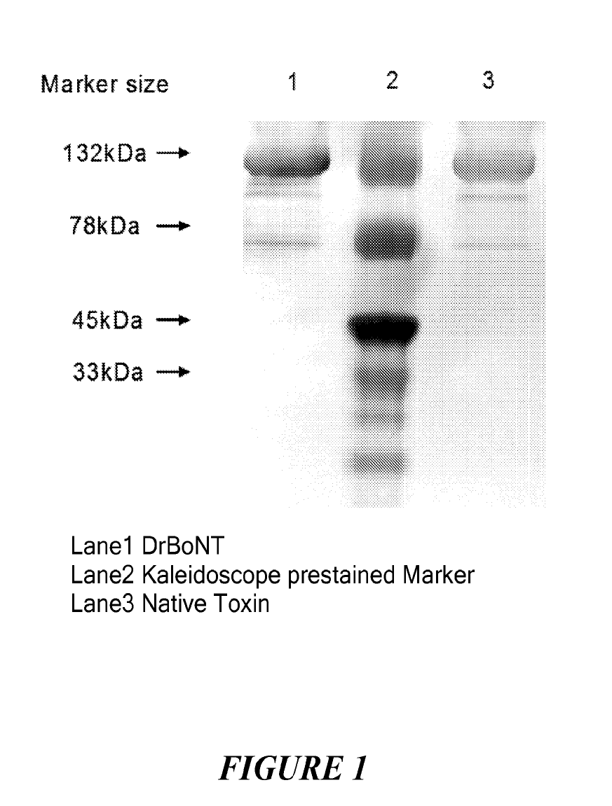

While both native BoNT/A and DR BoNT/A show very strong reactions with anti-BoNT/A antibody, native BoNT/E shows little or no reactivity. This observation indicates that the anti-BoNT/A antibody utilized herein was specific to BoNT/A. Since Western blot analysis confirms that anti-BoNT/A antibody reacts equally against DR BoNT/A and BoNT/A, one may conclude that these two botulinum proteins expose similar epitopes. See, FIG. 1.

2. Isoelectric Focusing

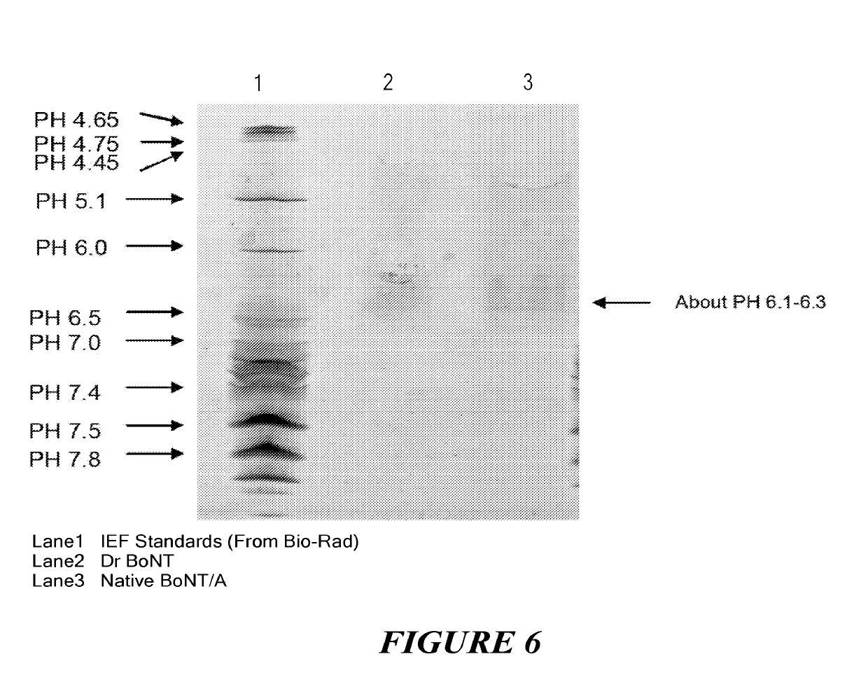

Native BoNT/A and DR BoNT/A were focused at the same position on an isoelectric (IEF) gel, wherein the pI for native BoNT/A and DR BoNT/A samples was estimated at 6.1-6.3. FIG. 6. This pI range corresponds well with theoretical pI estimates of 6.3 for DR BoNT/A predicted by Expasy® software. This observation indicates that native BoNT/A and DR BoNT/A not only have similar amino acid compositions, but their secondary and tertiary folding structure is also similar (infra).

3. Endopeptidase Activity

SNAP-25-GST tagged protein has been shown to be a substrate of BoNT/A. Sharma et al., Biochemistry 43:4791-4798 (2004). Reduction of the disulfide bond between the light and heavy chains is required for initiation of endopeptidase activity so 1 mM DTT may be added to the reaction mixture to achieve optimum enzyme activity. Cai et al., (1999) Biochemistry 38:6903-6910.

The BoNT/A active site utilizes a zinc ion (Zn²⁺) to perform its endopeptidase catalytic activity. Although it is not necessary to understand the mechanism of an invention, it is believed that the E262 residue directly coordinates the hydrogen bonding of the zinc to relatively nucleophilic water molecules. Li et al., Biochemistry 39:2399-2405 (2000).

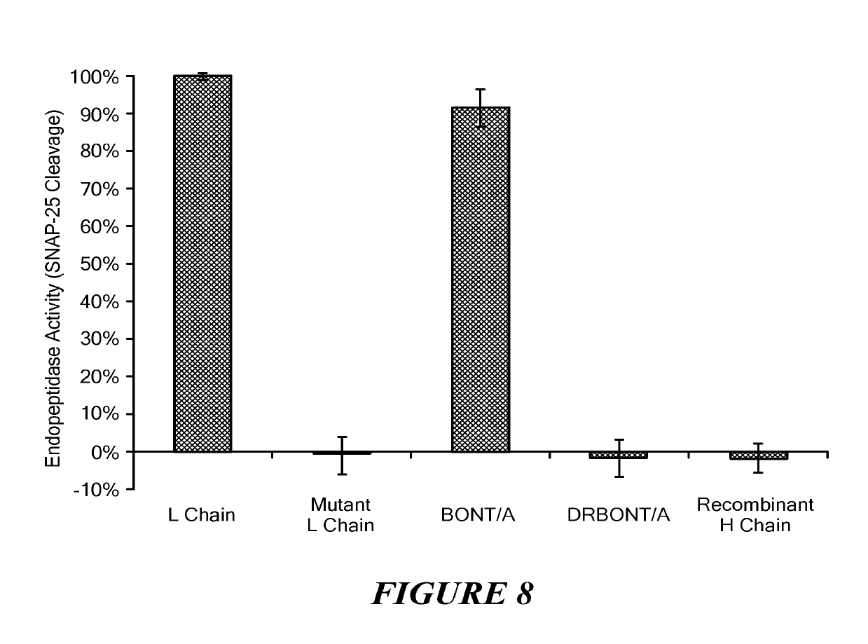

Endopeptidase activity of a double mutant BoNT/A L chain, DR BoNT/A protein, and a recombinant BoNT(HC)/A was found to be negligible against SNAP-25 as compared to a native BoNT/A and/or a wild-type BoNT(LC)/A. FIG. 8. These results show that BoNT/A endopeptidase activity can be removed completely through mutations within the LC, and that the BoNT(HC)/A has no endopeptidase activity. Nonetheless, mutations in either the HC and/or LC can result in abolishing most or all BoNT/A endopeptidase activity. The E224A→E262A mutation removed almost all BoNT/A endopeptidase activity in contrast to native BoNT/A and recombinant LC/A wild type endopeptidase activity.

Further, triple and/or quadruple DR BoNT/A mutants are also devoid of endopeptidase activity. (data not shown). Although it is not necessary to understand the mechanism of an invention, it is believed that once the double mutant reforms the catalytic site, further mutation does not reform the active site.

The present invention contemplates a plurality of BoNT/A mutants in relation to the wild type BoNT/A sequence:

a) Wild Type BoNT/A: H²²³-E²²⁴-L²²⁵-I²²⁶-H²²⁷+E²⁶²

b) DR BoNT/A: H²²³-A²²⁴-L²²⁵-I²²⁶-H²²⁷+A²⁶²

c) DR BoNT/A-T M²²³-A²²⁴-L²²⁵-I²²⁶-H²²⁷+A²⁶²

d) DR BoNT/A-Q M²²³-A²²⁴-L²²⁵-I²²⁶-Q²²⁷+A²⁶²

In one embodiment, a double BoNT/A mutant comprises DR BoNT/A. In one embodiment, a triple BoNT/A mutant comprises DR BoNT/A-T. In one embodiment, a quadruple BoNT/A mutant comprises DR BoNT/A-Q.

4. Protein Folding

Quite often recombinant proteins expressed in E. coli do not fold properly into native conformation due to several reasons. For example, the absence of appropriate and compatible chaperones is believed to interfere with proper protein folding. Protein folding may also be affected during extraction and purification procedures. BoNT/A L chain and DR BoNT/A are adequately soluble in aqueous solution such that they are present in the soluble fraction of a bacterial extract. Recombinant H chain (i.e., for example, following E. coli expression), however, forms inclusion bodies that requires harsher treatment for its extraction and purification.

a. Secondary Structure Folding

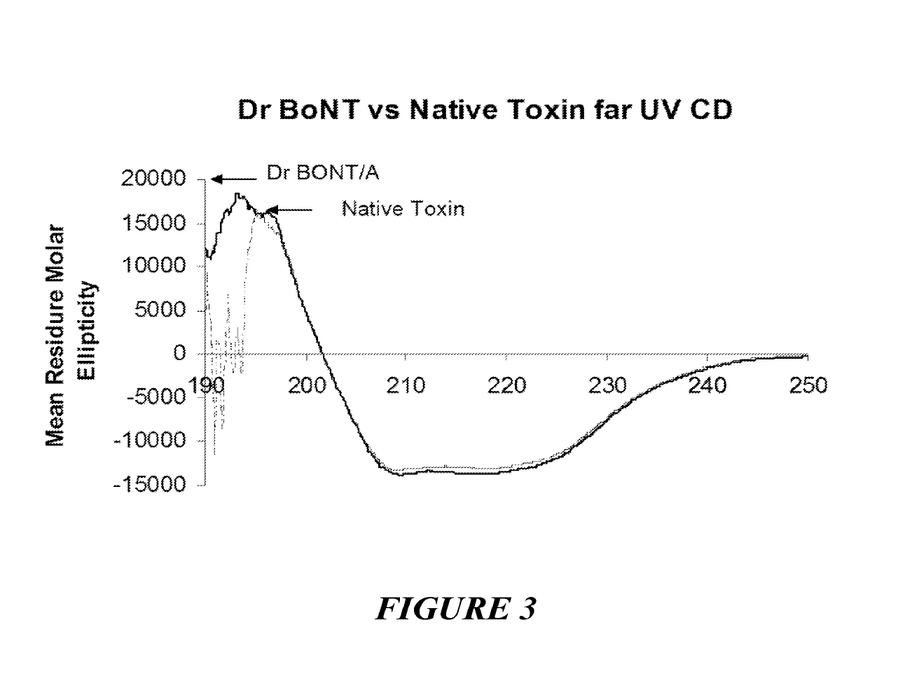

Circular dichroism was employed to compare DR BoNT/A and native BoNT/A protein folding. The CD spectra of DR BoNT/A is virtually identical to native BoNT/A, showing that the secondary structure folding of recombinant DR BoNT/A has not been affected by the mutations. For example, DR BoNT/A and native BoNT/A have two strong absorption maxima at 208 and 222 nm. The signal difference below 200 nm is due to the saturation of the PMT due to the presence of excessive salt. See, FIG. 3. Although it is not necessary to understand the mechanism of an invention, it is believed that with only two amino acid residues mutated, and the folding virtually same as the native BoNT/A.

b. Tertiary Structure Folding

In order to assess the tertiary structure folding of DR BoNT/A, its trypsin digestion pattern was compared with that of native BoNT/A. Two different concentrations of trypsin were used to digest DR BoNT/A. The protein concentration for both DR BoNT/A and native BoNT/A was approximately 0.5-0.6 mg/ml. The ratio between protein and trypsin was either 250:1 or 50:1 (w/w). Trypsin digestion was carried out in 50 mM Tris buffer, pH 7.6, containing 200 mM NaCl and 5 mM CaCl₂ at room temperature (25° C.) for various periods (5, 10, 30, and 60 min) of incubation. After a given incubation period 20 μl digested mixture was taken out, and 1 mM PMSF was added to it, before boiling it for 5 min with SDS-PAGE loading buffer. Sharma et al., 1998 (supra).

When the trypsin concentration used was low (i.e., for example, at a ratio of protein to enzyme of 250:1), DR BoNT/A was mostly digested into LC and HC. Although it is not necessary to understand the mechanism of an invention, it is believed that DR BoNT is a single peptide, and trypsin nicks it at the same position as in a native BoNT/A (i.e., for example, between amino acid residue 448 and 449).

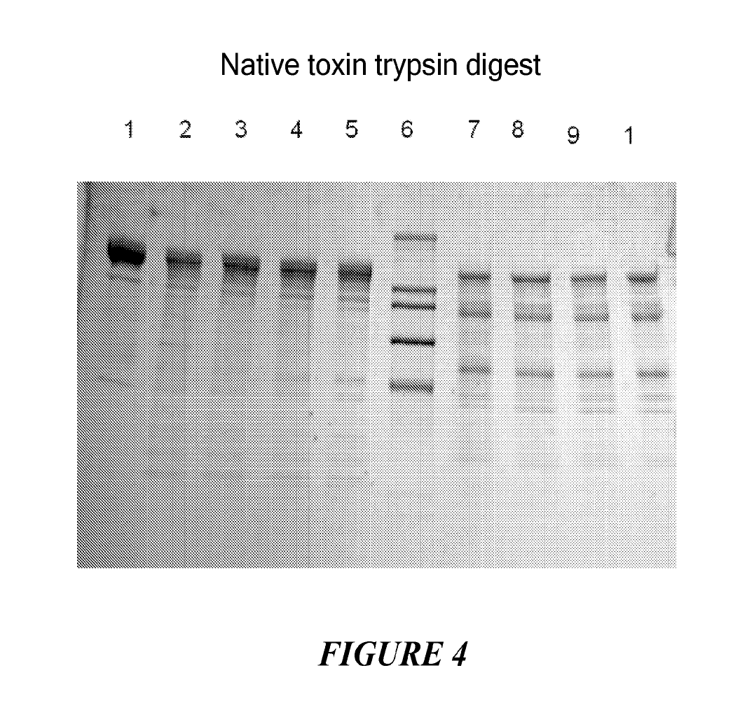

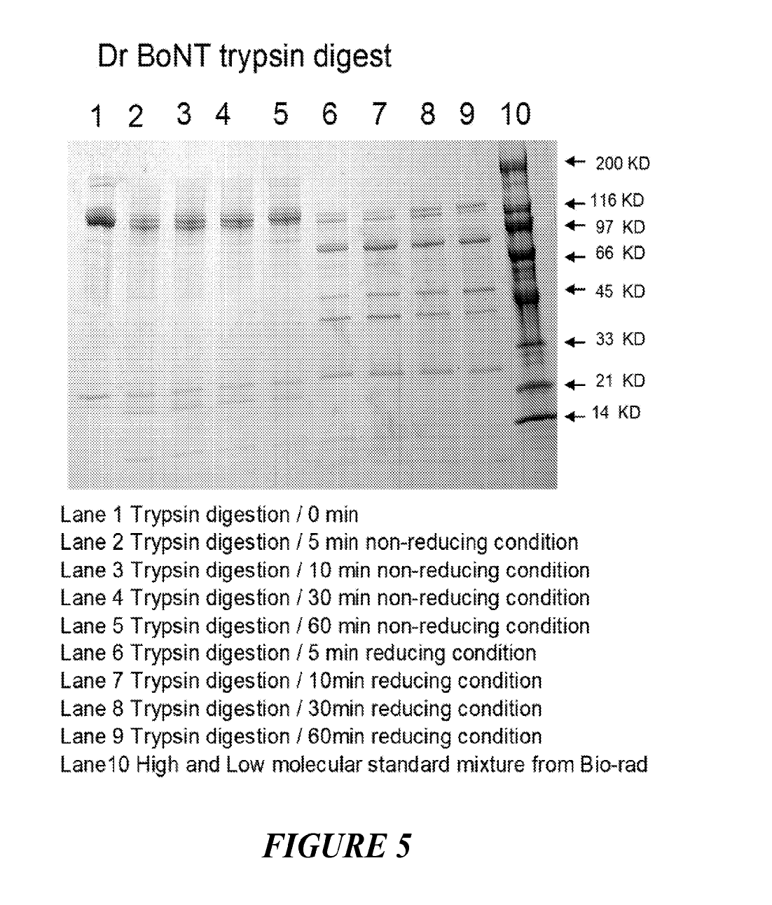

A comparison of the tertiary folding pattern of DR BoNT/A and native BoNT/A was prepared using a 50:1 (BoNT/A:trypsin, w/w) ratio and digested for a varying periods of time (i.e., for example, 5, 10, 30, and 60 min). Thereafter, the digested samples were boiled in non-reducing and/or reducing SDS-PAGE-loading buffer for 5 min. A SDS-PAGE analysis showed very similar patterns for native and DR BoNT/A. See, FIG. 4 and FIG. 5, respectively. DR BoNT/A disulfide bonding were formed correctly since the banding patterns were the same as for native BoNT/A under reducing and non-reducing conditions. Additionally, since the proteolytic fragmentation after trypsinization is similar in both DR BoNT/A and native BoNT/A, tertiary structure folding is also expected to be the same, indicating that DR BoNT/A folding corresponds to that of the native BoNT/A.

B. DR BoNT/A Created by Fusion Proteins

In one embodiment, the present invention contemplates a BoNT-C3E (B-C3E) chimera protein comprising a BoNT fragment and C3E fragment. In one embodiment, the BoNT fragment lacks an endopeptidase domain. In one embodiment, the BoNT-C3E chimera is non-toxic. In one embodiment, the BoNT fragment comprises a BoNT/A fragment. In one embodiment, the C3E fragment replaced the endopeptidase domain. In one embodiment, the C3E fragment comprises ADP-ribosyltransferase activity. Although it is not necessary to understand the mechanism of an invention, it is believed that the non-toxic BoNT/A-C3E chimera protein lacks endopeptidase activity, retains neurospecific receptor binding affinity and cell membrane translocation competency (e.g., for therapeutic cargo access to endosomal vesicles). It is further believed that non-toxic BoNT/A-C3E chimera proteins carry the C3E fragment as cargo, thereby encompassing a functional therapeutic delivery module comprising a functional C3E ADP-ribosyltransferase domain.

In some embodiments, the present invention contemplates methods for the delivery of a therapeutically efficacious BoNT-C3E chimera that targets the RhoA signaling pathway thereby resulting in axonal regenerative therapy (i.e., for example, SCI therapy). Although it is not necessary to understand the mechanism of an invention, it is believed that these methods exploit BoNT-C3E chimera protein structure, function and trafficking potentials. The data presented herein demonstrate an ability of a non-toxic botulinum neurotoxin derived fragment comprising receptor binding domains and/or cell membrane translocation domains, to deliver a therapeutic cargo into neuronal cells (e.g., injured axon cells).

In one embodiment, the present invention contemplates an improvement to C3E spinal cord treatment technology by developing a C3E exoenzyme/neurotoxin chimeric protein. (i.e., for example, a BoNT(HC)/A-C3E chimera). In some embodiments, the present invention contemplates C3E-exoenzyme chimeric proteins comprising a non-toxic BoNT/A fragment. In one embodiment, the non-toxic BoNT/A fragment lacks an endopeptidase domain. In one embodiment, the non-toxic BoNT/A fragment comprises at least one mutation. In one embodiment, the non-toxic BoNT/A fragment comprises at least two mutations.

BoNTs are distinct proteins from C3E proteins, although both are produced by C. Botulinum. BoNTs are highly efficient in their ability to target peripheral nerve terminals at neuromuscular junctions at extremely minuscule doses following systemic intoxication or subsequent to a localized intramuscular injection. A native BoNT mode of action exerts toxicity and/or therapeutic activity by blocking neurotransmitter release upon cell receptor binding and translocation into pre-synaptic membrane terminals. BoNTs consist of three structurally and functionally distinct domains involved in receptor binding at pre-synaptic membrane terminals, endosomal translocation, and an enzymatic domain that confers toxicity by blocking the neurotransmitter release by its endopeptidase activity.

In some embodiments, the present invention contemplates a method for using a DR BoNT/A-based C3E fusion chimeric protein to deliver C3E specifically into neuronal cells for axonal regeneration in SCI therapy. Structurally and functionally distinct domains in BoNT/A provide an opportunity to engineer BoNT chimera by recombinant DNA technology, and take advantage of BoNT biological features and physiology.

For example, a DR BoNT/A chimeric protein delivery vehicle that is specifically targeted to neuronal cells may be constructed by replacing a BoNT endopeptidase domain with a C3E-ADP-ribosyltransferase (e.g., a C3E exoenzyme), while retaining other native domains of BoNT/A as a fusion/chimeric protein. See, FIGS. 12A and 12B. In one embodiment, an intact receptor binding domain (HC2) confers neurospecific targeting. In one embodiment, an intact translocation domain (HN) containing a belt region, preserves competence for channel formation and physical disassociation of C3E into the neuronal cytosol. Although it is not necessary to understand the mechanism of an invention, it is believed that C3E dissociation is mediated by intracellular proteases. Other structural aspects ensure the formation of disulfide bridge between the C3E and HC, efficient internalization and/or intracellular release of therapeutic cargo molecules. For example, C3E, which does not have cell receptor binding properties, is known to be translocation-competent across acidic vesicles like other cargoes of the A-B group binary toxins, when heterologously fused to cell binding components that undergo receptor mediated endocytosis. Marvaud et al., “Clostridium perfringens IOTA toxin: Mapping of the Ia domain involved in docking with Ib and cellular and internalization” J. Biol. Chem. 277:43659-43666 (2002).

The enzyme C3E, upon cytosolic delivery was also show to have RhoA ADP-ribosylation activity (data not shown), thereby obviating any potential unfolding and refolding issues and/or variable pH compartments occurring after the translocation step.

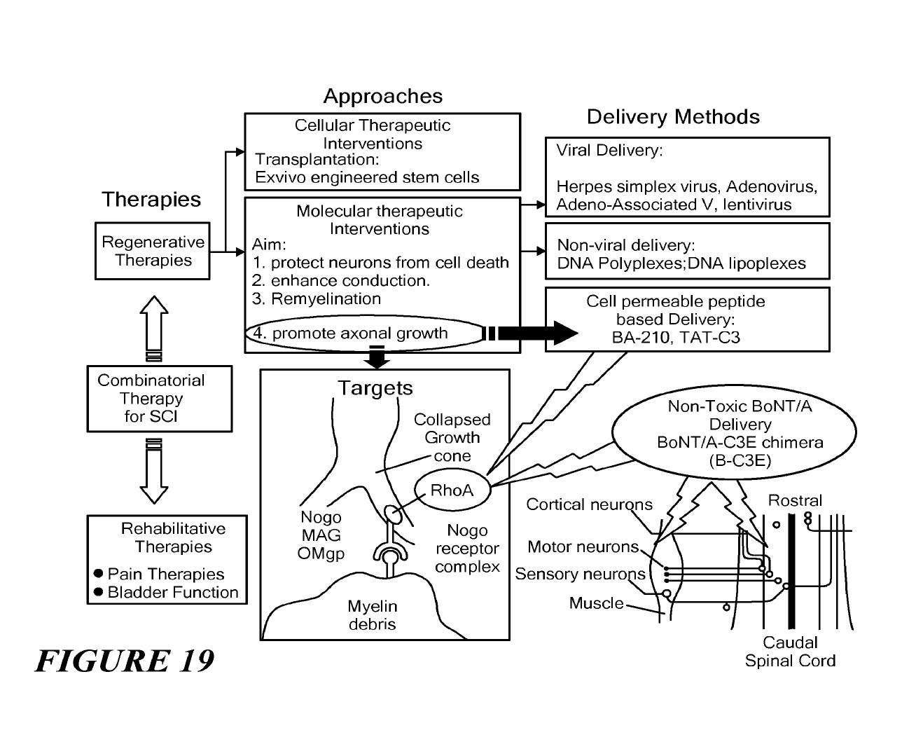

A chimeric DR BoNT/A-C3E fusion chimeric protein without endopeptidase activity is rendered non-toxic, but retains ADP-ribosyltransferase catalytic activity. Overall therapeutic interventions, delivery methods and strategic pathways are schematically presented herein. See, FIG. 19.

Although it is not necessary to understand the mechanism of an invention, it is believed that some of the chimeras described herein result from fusing C3E exoenzyme with the Heavy Chain component of Botulinum Neurotoxin (BoNT(HC)) by recombinant DNA technology. It is further believed that a BoNT(HC)-C3E chimera has several advantages over the currently available cell-permeable C3E product (BA210) that is currently under clinical trial.

1. Spinal Cord Injury (SCI) and RhoA Target Pathways

In some embodiments, the BoNT/C3E chimeras modulate the RhoA system. RhoA is believed to be abnormally activated in injured axons distal from the lesion site of SCI, leading to rigidification of the actin cytoskeleton, thereby causing growth cone collapse, inhibition of axonal elongation, and cell body rounding. Thus RhoA, an intracellular GTP-binding protein, becomes the central converging point of different signaling mechanisms of different inhibitory cues during axonal injuries. Cellular events occurring during an acute phase of SCI typically include, but are not limited to, post-traumatic necrosis of severed neurons and non-neuronal cells, reactive glial scar formation, wallerian degeneration of injured neuron involving structural destruction of axolemma, dismantled axonal cytoskeleton at the distal end, and myelin sheath breakage. Myelin-derived molecules, Nogo, myelin-associated glycoprotein (MAG), OMgp, and ephrinB3, as well as the astrocyte-scar-enriched chondroitin sulfate proteoglycans (CSPGs) are believed to be inhibitory molecules against post-SCI axon regeneration (2,3). Interestingly, many myelin derived inhibitors signal through a common receptor complex (e.g., NgR, p75/TROY and Lingo complex) to activate RhoA. Although less understood the CSPGs are also though to employ the RhoA signaling mechanism.

2. RhoA is a Targetable Biotherapeutic for Axonal Regeneration in SCI

Molecular therapies for SCI aim to modulate neuron survival, neurite outgrowth, or to enhance synaptic plasticity and neurotransmission. Consequently, one molecular therapeutic approach to promote axonal regeneration after SCI may be by targeting intrinsic molecules like RhoA. It has been reported that Clostridium botulinum C3 Exoenzyme (C3E) protein irreversibly modifies the RhoA effector domain by ADP-ribosylation at asparagine-41. Recently, a cell permeable, C3E fusogenic derivative, C3-05 (or BA-205) has been developed and is currently under human clinical trials for axonal growth restoration after SCI.

3. An Effective and Safe C3E Drug Delivery System is Currently Unmet

Development of a neuron-selective C3E delivery system compatible with SCI pathology had not been previously attempted. Although it is not necessary to understand the mechanism of an invention, it is believed that the BoNT/C3E chimeras described herein efficiently deliver C3E due to inherent resistance against acidified endosomal vesicles. It is further believed that other non-viral strategies are sensitive to these acidic endosomal vesicles resulting poor translocation efficiencies.

Some current major limitations in the existing methods and strategies to deliver therapeutic drugs like C3E include, but are not limited to;

i) Non-Specific Delivery and Toxicity

C3E does not have natural cell binding component to allow efficient entry mechanisms specifically into neuronal cells. Consequently, the use of cell-permeable C3E variants like C3-05 (or BA-205) also allow the unwanted, and deleterious, non-specific entry of C3E into the glial cells at the lesion site. One impact of this side effect would be a perturbation of the necessary synergistic relationship between the major types of glial cells. Recent studies have indeed shown that RhoA ADP-ribosylation by C3E and Rho-kinase inhibition in astrocytes enhanced the expression of neurite-growth inhibitory chondroitin sulfate proteoglycans within the extracellular matrix (8), and also induced proinflammatory response in microglia, mediated by NFκB. These unwanted effects of cell-permeable C3E variants could negatively impact axon regeneration. However, the BoNT/C3E chimeras as described herein may effectively target only the neuronal cells of the severed spinal cord, when applied to the lesion site, as opposed to indirect side effects on glial cells. Such specificity and selectivity of RhoA targeting in neuronal population without their entry into glial cells might reduce the toxicity of the drug and may effectively promote the axon regeneration.

ii) Route of Delivery

The current human clinical trials with BA-205 are limited to topical application on the lesion site. In contrast, minimally invasive routes like intramuscular administration are of extremely interest, and a superior route of delivery for SCI therapy.

iii) Limitations in Other Viral and Non-Viral Choice of Carriers

Viral vectors involving gene therapies suffer with local immune responses, and safety issues, while non-viral carriers suffer from low transfection efficiencies, as they undergo internalization in the vesicles that are part of endo-/lysosomal pathway and are unable to escape from the acidifying endosomal compartments in neurons. So there is a critical need to develop neurotargeting capacities for drugs like C3E, which could be administered through minimally invasive routes, without perturbing the normal physiology of non-neuronal cells surrounding the severed neuron during SCI. Current clinical trials with C3E using fibrin sealant formulation are aimed for topical application on the lesion site. Although minimally invasive or traumatic routes are preferable in account to the limited number of motor neuron availability, non-viral carriers had not yet been tested for C3E or any other drug delivery in context to SCI. Among less invasive routes, intramuscular administration using retrograde transporting carriers are of extremely interest to use in SCI therapy. The BoNT/C3E chimeras described herein provide such an advantage of having an intramuscular drug for axon regeneration during SCI.

III. Therapeutic Delivery by Botulinum Neurotoxin Heavy Chain Variants

A. Generic Therapeutic Delivery Vehicle (DDV)

A notable feature of Botulinum Neurotoxin Heavy Chain variants (BoNT(HC)) being a specific delivery vehicle is its ability to deliver cargo other than own LCs, as demonstrated by heterologous LC delivery. Bandyopadhyay et al., “Role of the heavy and light chains of botulinum neurotoxin in neuromuscular paralysis” J. Biol. Chem. 262:2660-2663 (1987). Other therapeutic cargo proteins (i.e., for example, luciferase, GFP, or dihydrofolate reductase) may be attached to the amino terminus of a full-length botulinum neurotoxin serotype-D without any light chain replacement and toxicity. Bade et al., “Botulinum neurotoxin type D enables cytosolic delivery of enzymatically active cargo proteins to neurons via unfolded translocation intermediates” J Neurochem. 91(6):1461-72 (2004). However, BoNT serotype D had not been extensively studied for retrograde properties and is less characterized in therapeutics than other BoNT serotypes.

A related tetanus toxin HC has been shown to deliver a gelonin protein into the cytosol as well as several passenger proteins into lower motor neurons. Johnstone et al., “The heavy chain of tetanus toxin can mediate the entry of cytotoxic gelonin into intact cells” FEBS Lett. 265:101-103 (1990); Benn et al., “Tetanus toxin fragment C fusion facilitates protein delivery to CNS neurons from cerebrospinal fluid in mice” J. Neurochem. 95:1118-1131 (2005); and Larsen et al., “A glial cell line-derived neurotrophic factor (GDNF): tetanus toxin fragment C protein conjugate improves delivery of GDNF to spinal cord motor neurons in mice” Brain Res. 1120:1-12 (2006). While the above studies imply that nontoxic, neuron-binding tetanus toxin fragment (TTC) of tetanus toxin, could be a potential non-viral neuronal delivery from animal studies, its translation into clinical practice for human therapy is not feasible due to the general vaccination of most of the population with tetanus toxoid (TT). Consequently, TeNT(HC) administration to patients with any kind of injuries, including SCI, would potentially generate interfering antibodies and interfere its efficacy as a delivery vehicle. It is now even recognized that TeNT(HC) mutants lacking immunodominant epitopes may not be a straightforward approach to remedy this disadvantage. Caleo et al., “Central effects of tetanus and botulinum neurotoxins” Toxicon 54:593-599 (2009).

In some embodiments, the present invention contemplates non-toxic versions of BoNT/A derivatives which have superior advantages over TeNT because: i) there is no concern of pre-existing immunity against BoNT/A, ii) botulism is a rare disease; iii) generally only occupational workers are vaccinated; and iv) therapeutic doses used for neuromuscular disorder treatments are extremely low generally avoiding systemic immune response. It has been recently demonstrated that recombinant BoNT(HC)/A delivered a dextran-dye cargo specifically to the neuronal cytosol as a generic drug delivery vehicle (DDV), upon being internalized via endocytosis similar to full-length holotoxin. Zhang et al., “An efficient drug delivery vehicle for botulism countermeasure” BMC Pharmacol. 27:9-12 (2009).

In one embodiment, the present invention contemplates a generic drug delivery vehicle (DDV) comprising a BoNT(HC)/A, wherein the vehicle lacks a BoNT(LC)/A. For example, a DDV construct comprises a targeting molecule, a Cy3 labeled rHCA, wherein a therapeutic molecule (i.e., for example, Oregon green 488 (OG488) labeled 10 kDa dextran) is linked to the vehicle by a disulfide bond. Goodnough et al., “Development of a delivery vehicle for intracellular transport of botulinum neurotoxin antagonists” FEBS Letters 513:163-168 (2002).

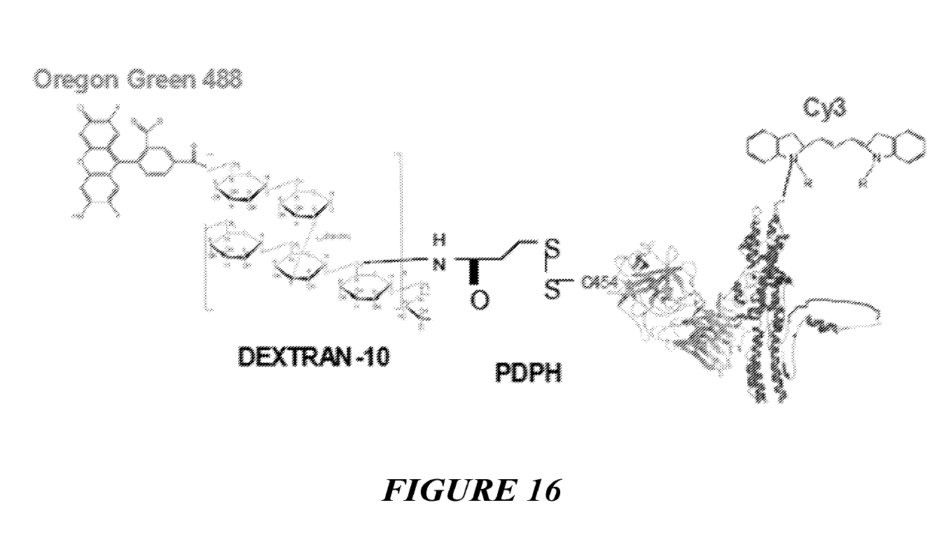

In one embodiment, a 3-(2-pyridyldithio)propionic acid hydrazide (PDPH) linker is bound to a DR BoNT(HC)/A cysteine sulfhydryl group. FIG. 16. In one embodiment, the cysteine group is C⁴⁵⁴. In one embodiment, Cy3 and Oregon green 488 are bound to an O-amino groups of lysine in the rHCA and dextran, respectively. In one embodiment, the dextran is conjugated to the HC/A by a C—N bond in one of the glucose residues. In one embodiment, the DDV is linked to multiple therapeutic molecules. In one embodiment, the DDV is attached to a dextran carrier for neuronal delivery.

B. Neuronal Specific Therapeutic Vehicle

BoNT's potential to undergo retrograde axonal transport and transcytosis to second order neurons in both CNS and peripheral circuits, provide opportunities to treat certain ailments of CNS to deliver it at hard-to-inject sites. Kuehn B. M., “Studies, reports say botulinum toxins may have effects beyond injection site” JAMA 299:2261-2263 (2008). Established intramuscular injection protocols of BoNTs in clinical therapy as a FDA approved drug, may provide minimally invasive delivery strategies for conditions like SCI.

Although it is not necessary to understand the mechanism of an invention, it is believed that a BoNT(HC)/A-C3E chimera induces axonal regenerative therapy of SCI by providing a non-viral C3E delivery system, wherein the BoNT(HC)/A portion blocks RhoA signaling. It is further believed that botulinum neurotoxin (BoNT) can exploit its ability to target neurons with extreme efficacy in humans, and may provide a non-toxic and effective drug delivery platform (e.g., for targeted delivery of C3E).

In some embodiments, the present invention contemplates the intramuscular administration of a DR BoNT(HC)/A-C3E chimera protein such that the chimera is internalized into an undamaged axon. In one embodiment, the chimera undergoes retrograde transport and/or neuronal transcytotic movement, wherein the chimera is delivered to the distal side of a severed axon. In one embodiment, the severed axon is in synaptic contact to the undamaged axon. Although it is not necessary to understand the mechanism of an invention, it is believed that chimera delivery through an undamaged axon has potential to confer neuronal regeneration activity by inhibiting RhoA in adjacent severed axons at lesion sites, thereby augmenting axonal regeneration and neuroprotection. Such modes of C3E delivery to block RhoA for axonal regenerative therapy could be successful within a reasonable therapeutic window, and can be tailored with other combinatorial therapies. This approach would not only be used as a treatment during the onset of acute or subacute stages after injury and before complete degeneration of the injured axon occurs at the distal side, but also be applied prophylactically in suspected axonal injuries due to ease of administration.

In one embodiment, the present invention contemplates creating non-toxic BoNTs and engineering chimeric protein fusions. In one embodiment, the non-toxic BoNT chimeric protein achieves an efficient neuronal delivery system for neuronal-targeted biological therapeutic delivery. In one embodiment, the biological therapeutic may be C3E, wherein injured axons are regenerated by blocking RhoA signaling. In one embodiment, the injured axons are a result of SCI.

1. Axonal Targeting of DR BoNT(HC)/A Variants

Data described herein demonstrate the selective entry into neuronal cells of a catalytically deactivated recombinant BoNT/A comprising an inactivated LC with at least two mutated active site residues or lacking an LC endopeptidase domain (DR BoNT(HC)/A).

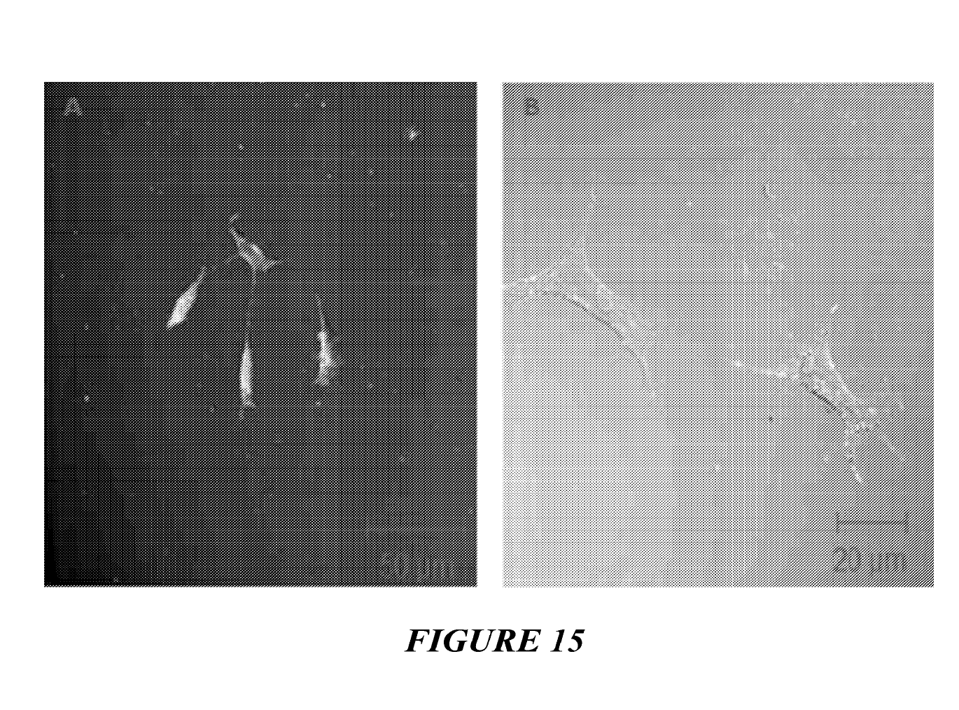

DR BoNT/A internalization into human neuroblastoma SH-SY5Y cells was measured using a dye labeled DR BoNT/A (Alexa-488, green) with non-neuronal human rhabdomyosarcoma cells (as internal controls) under a laser scanning confocal microscopy. Plasma membranes were specifically labeled by staining cells with Wheat Germ Agglutinin (WGA)-Alexa 594 (red). Cells were incubated for 2 h at 37° C. with Alexa-488 labeled DrBoNT/A. Merged images obtained by treating SH-SY5Y (FIG. 15A) and human rhabdomyosarcoma (FIG. 15B) (green), counter stained by the WGA-Alexa 594 (red).

These data show that: (i) DR BoNT(HC)/A selectively binds to neuronal cells as compared to rhabdomyosarcoma muscle cells, as revealed substantially stronger labeling of neuronal cells; (ii) with SH-SY5Y cells (panel A) the internalization of Alexa-488 labeled DR BoNT(HC)/A (green) inside the cell membrane compartment, counter stained by the WGA-Alexa 594 (red) was observed while in the human rhabdomyosarcoma (panel B) the labeled DrBoNT(HC)/A is mostly localized to the cell surface without being internalized. Although it is not necessary to understand the mechanism of an invention, it is believed that such cell selectivity is highly advantageous for neuronal targeting and drug delivery as contemplated in some embodiments described herein.

### IV. Botulinum Toxin Clinical Formulations and Administration

Therapeutic administration of DR BoNT variants are usually through local intramuscular injections. When free-¹²⁵I-BoNT/A is injected intramuscularly, there were no detectable systemic effects or generalized botulinum neurotoxin toxicity in either rats or rabbits, since they remained at the injection site with no significant diffusion and almost no radioactivity was recovered from the brain. Tang-Liu et al., “Intramuscular injection of ¹²⁵I-botulinum neurotoxin-complex versus ¹²⁵I-botulinum-free neurotoxin: time course of tissue distribution” Toxicon 42:461-469 (2003). Administration of DR BoNT variants are expected to have similar pharmacokinetics of FDA-approved therapeutic formulations containing botulinum neurotoxins, since only the LC enzymatic domain of a BoNT peptide is engineered, retaining the other HC determinants involved in binding and trafficking. The administration of DR BoNT variants could be achieved by similar intramuscular route of administration close to the site of spinal cord injury for axonal regenerative therapy. This obviates toxicity issues of DR BoNT variants targeting RhoA in healthy neurons beyond the site of injury since DR BoNT variants only have limited transynaptic movements at lower concentrations and exert biological effects only to the local nerve terminals.

However, if a DR BoNT variant had to be administered at distant sites to the spinal cord lesion, transynaptic movement across the nerve could be still facilitated by applying higher doses. This is an advantage of some present embodiments due to loss of toxicity by replacing endopeptidase activity with a C3E exoenzyme. Also, the retained structural determinants in some DR BoNT-C3E chimeras, basically a heavy chain (HC/A) composed of RBD and TD are not toxic up to 100,000 LD₅₀ in mice as compared to wild type BoNT/A. Although it is not necessary to understand the mechanism of an invention, it is believed that these considerations facilitate the retrograde and trans-synaptic movement of the chimera across the nerve, when given through minimally invasive intramuscular route of administration, distant to the site of injury.

In one embodiment, the present invention contemplates a drug delivery system comprising a non-toxic botulinum HC chain. In one embodiment, the HC is attached to a liposome, wherein the liposome comprises a therapeutic drug. In one embodiment, the therapeutic drug is effective against SCI. Although it is not necessary to understand the mechanism of an invention, it is believed that a DR BoNT/A peptide contains an intact light chain component, and thus can be used as a targeted drug delivery system to cells comprising BoNT/A HC receptors. Further, a BoNT(HC)/A targeted drug delivery system (i.e., a liposome) can provide local administration of therapeutic drugs (i.e., for example, anti-inflammatory drugs).

The present invention contemplates several drug delivery systems to which a DR BoNT(HC)/A may be attached that provide for roughly uniform distribution, have controllable rates of release and may be administered by a variety of different routes. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a liposome medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In one embodiment, the present invention contemplates a medical device comprising several components including, but not limited to, a reservoir comprising a carrier comprising a non-toxic BoNT/A H chain, a catheter, a sprayer, and/or a tube. In one embodiment, said medical device administers the carrier either internally or externally to a patient.

One embodiment of the present invention contemplates a drug delivery system comprising at least one pharmaceutical drug effective against a botulinum intoxication and/or secondary conditions thereof. Such pharmaceutical drugs may include, but are not limited to, anti-inflammatory, corticosteroid, antithrombotic, antibiotic, antifungal, antiviral, analgesic and anesthetic drugs. In one embodiment, the drug includes, but is not limited to, peptides, proteins, polypeptides and/or fragments thereof. In one embodiment, the drug includes, but is not limited to, nucleic acids, polynucleic acids and/or fragments thereof. In one embodiment, the nucleic acid comprises silencing RNA (siRNA). In one embodiment, the nucleic acid comprises interfering RNA (RNAi). In one embodiment, the polynucleic acid comprises a sense nucleic acid sequence. In one embodiment, the polynucleic acid comprises an antisense nucleic acid sequence.

Microparticles

In one embodiment, the present invention contemplates a medium comprising a microparticle, wherein the microparticle has an attached DR BoNT/A H chain. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Liposomes

In one embodiment, the present invention contemplates liposomes capable of attaching a DR BoNT/A H chain. Liposomes are microscopic spherical lipid bilayers surrounding an aqueous core that are made from amphiphilic molecules such as phospholipids. For example, a liposome may trap various pharmaceutical agents between their hydrophobic tails of the phospholipid micelle. Water soluble drugs can be entrapped in the core and lipid-soluble drugs and/or dissolved in the shell-like bilayer. Liposomes have a special characteristic in that they enable water soluble and water insoluble chemicals to be used together in a medium without the use of surfactants or other emulsifiers. Further, liposomes may form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds (i.e., a pharmaceutical agent). In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

The present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes attached to a DR BoNT/A H chain that provide controlled release of a pharmaceutical agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

Liposome compositions can be broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel. As used herein, the terms “microspheres, microcapsules and microparticles” (i.e., measured in terms of micrometers) are synonymous with their respective counterparts “nanospheres, nanocapsules and nanoparticles” (i.e., measured in terms of nanometers). Further, the terms “micro/nanosphere, micro/nanocapsule and micro/nanoparticle” are also used interchangeably.

Microspheres are obtainable commercially (Prolease®, Alkerme's: Cambridge, Mass.). For example, a freeze dried DR BoNT/A medium is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of drug release. Miller et al., Degradation Rates of Oral Resorbable Implants {Polylactates and Polyglycolates: Rate Modification and Changes in PLA/PGA Copolymer Ratios, J. Biomed. Mater. Res., Vol. II: 711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of DR BoNT/A is added to the biodegradable polymer metal salt solution. The weight ratio of DR BoNT/A to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and DR BoNT/A is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and DR BoNT/A mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a pharmaceutical agent for a duration of approximately between 1 day and 6 months. Controlled release microcapsules may be produced by using known encapsulation techniques such as centrifugal extrusion, pan coating and air suspension. Microspheres/microcapsules can be engineered to achieve particular release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere control the drug release rate such that custom-designed microspheres are possible, including effective management of the burst effect. ProMaxx® (Epic Therapeutics, Inc.) is a protein-matrix drug delivery system. The system is aqueous in nature and is adaptable to standard pharmaceutical drug delivery models. In particular, ProMaxx® are bioerodible protein microspheres that deliver both small and macromolecular drugs, and may be customized regarding both microsphere size and desired drug release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

One embodiment of the present invention contemplates microspheres or microcapsules attached to a DR BoNT/A H chain comprising a pharmaceutical agent. Such pharmaceutical agents include, but are not limited to, anti-inflammatory, corticosteroid, antithrombotic, antibiotic, antifungal, antiviral, analgesic and anesthetic.

In one embodiment, a microparticle contemplated by this invention comprises a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a DR BoNT/A H chain may be directly bound to the surface of the microparticle and/or is indirectly attached using a “bridge” or “spacer”. The amino groups of a gelatin lysine group are easily derivatized to provide direct coupling sites. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

### V. Botulinum Pharmacokinetic Analysis

In one embodiment, the present invention contemplates a method of determining BoNT/A pharmacokinetics. The ability to accurately determine the distribution, bioavailability and elimination of a toxic substance in a body is hampered by the ethical considerations of administering a harmful compound. Consequently, limited knowledge is available regarding the pharmacokinetic parameters of botulinum toxin.

A detoxified botulinum toxin that maintains the same physical characteristics (i.e., for example, amino acid sequence and protein folding parameters) as native botulinum toxin would make an ideal candidate for use for pharmacokinetic analysis. In one embodiment, the present invention contemplates a method utilizing DR BoNT/A as a pharmacokinetic marker. Although it is not necessary to understand the mechanism of an invention, it is believed that DR BoNT/A will be distributed and eliminated from a body in an identical fashion as native BoNT/A.

Data describing drug modeling and pharmacokinetics are routinely obtained from standard clinical studies. For example, two comparable, open-label, randomized, parallel, placebo-controlled group comprising healthy volunteers may be utilized. In one group, DR BoNT/A may be administered as eight single IM doses: 300, 450, 600, 900, 1200, 1350, 1800, 2400 IU/kg. In a second group, DR BoNT/A may be administered as multiple dosage regimens: 150 IU/kg three times a week for four weeks and 600 IU/kg one per week for four weeks. Each treatment group may range in size but a minimum of at least 5 subjects is preferred.

Baseline DR BoNT/A concentrations for each subject are determined by averaging the predose values (10, 20 and 30 min). This value is then subtracted from the post-dose values at each time point to obtain the corrected serum DR BoNT/A concentrations. The mean of the corrected concentrations for all subjects is used for data analysis. Any measurement below the limit of assay detection should not be used as a data point.

Intravenous bolus administration can provide preliminary analysis to establish the appropriate compartment analysis. For example, if a one-compartment model is found to be adequate, the disposition of DR BoNT/A may be nonlinear mainly because of a dose-dependent decrease in clearance. See, Macdougall et al., Clin. Pharmacokinet. 20:99-113 (1991). In this case, a Michaelis-Menten function can be used to describe DR BoNT/A disposition. The IV data for DR BoNT/A concentrations (C_{DR BoNT/A}=Ap/Vd) versus time were fitted with the following equation:

$$\frac{d Ap}{d t} = - ( \frac{V max}{Km x Vd + Ap} ) Ap$$

where Aρ is the amount of DR BoNT/A in the body, Vmax is the capacity of the process, Km is the affinity constant or the plasma DR BoNT/A concentration at which the elimination rate reaches one-half Vmax, and Vd is the volume of distribution. The IV concentration-time profiles for the various doses of DR BoNT/A would be expected to fit a one-compartment model with non-linear disposition because, as a protein, DR BoNT/A can be expected to be restricted to the intravascular compartment. However, alternative pharmacokinetic compartment model fittings may be performed using commercially available software (i.e., for example, ADAPT II® software (see, e.g., Argenio et al., 1998. ADAPT II User's Guide, Biomedical Simulations Resource, University of Southern California, Los Angeles).

### VI. Detection of Botulinum Toxin

The invention contemplates detecting a bacterial toxin in a sample. The term “sample” in the present specification and claims is used in its broadest sense. On the one hand it is meant to include a specimen or culture. On the other hand, it is meant to include both biological and environmental samples.

Biological samples may be animal, including human, fluid, solid (i.e., for example, stool) or tissue; liquid and solid food products and ingredients such as dairy items, vegetables, meat and meat by-products, and waste. Environmental samples include environmental material such as surface matter, soil, water and industrial samples, as well as samples obtained from food and dairy processing instruments, apparatus, equipment, disposable and non-disposable items. These examples are not to be construed as limiting the sample types applicable to the present invention.

The invention contemplates detecting bacterial toxin by a competitive immunoassay method that utilizes recombinant toxin A and toxin B proteins, antibodies raised against recombinant bacterial toxin proteins. A fixed amount of the recombinant toxin proteins are immobilized to a solid support (e.g., a microtiter plate) followed by the addition of a biological sample suspected of containing a bacterial toxin. The biological sample is first mixed with affinity-purified or PEG fractionated antibodies directed against the recombinant toxin protein. A reporter reagent is then added which is capable of detecting the presence of antibody bound to the immobilized toxin protein. The reporter substance may comprise an antibody with binding specificity for the antitoxin attached to a molecule which is used to identify the presence of the reporter substance. If toxin is present in the sample, this toxin will compete with the immobilized recombinant toxin protein for binding to the anti-recombinant antibody thereby reducing the signal obtained following the addition of the reporter reagent. A control is employed where the antibody is not mixed with the sample. This gives the highest (or reference) signal.

The invention also contemplates detecting bacterial toxin by a “sandwich” immunoassay method that utilizes antibodies directed against recombinant bacterial toxin proteins. Affinity-purified antibodies directed against recombinant bacterial toxin proteins are immobilized to a solid support (e.g., microtiter plates). Biological samples suspected of containing bacterial toxins are then added followed by a washing step to remove substantially all unbound antitoxin. The biological sample is next exposed to the reporter substance, which binds to antitoxin and is then washed free of substantially all unbound reporter substance. The reporter substance may comprise an antibody with binding specificity for the antitoxin attached to a molecule which is used to identify the presence of the reporter substance. Identification of the reporter substance in the biological tissue indicates the presence of the bacterial toxin.

It is also contemplated that bacterial toxin be detected by pouring liquids (e.g., soups and other fluid foods and feeds including nutritional supplements for humans and other animals) over immobilized antibody which is directed against the bacterial toxin. It is contemplated that the immobilized antibody will be present in or on such supports as cartridges, columns, beads, or any other solid support medium. In one embodiment, following the exposure of the liquid to the immobilized antibody, unbound toxin is substantially removed by washing. The exposure of the liquid is then exposed to a reporter substance which detects the presence of bound toxin. In a preferred embodiment the reporter substance is an enzyme, fluorescent dye, or radioactive compound attached to an antibody which is directed against the toxin (i.e., in a “sandwich” immunoassay). It is also contemplated that the detection system will be developed as necessary (e.g., the addition of enzyme substrate in enzyme systems; observation using fluorescent light for fluorescent dye systems; and quantitation of radioactivity for radioactive systems).

### EXPERIMENTAL

The following examples serve to illustrate certain preferred embodiments and aspects of the present invention and are not to be construed as limiting the scope thereof.

### Example I

### Designing and Construction of E224A/E262A BoNT/A Plasmid

Genomic DNA of BoNT/A was isolated from C. botulinum Hall strain. The strategy to construct the full length BoNT/A mutant was to fuse BoNT/A-HC into the LC which is already cloned into the pBN3 vector between the EcoRI and PstI sites. The plasmid pBN3 harboring E224A/E262A BoNT/A-LC gene was first constructed based on the E224A single mutant LC constructed previously (Li et al., Biochemistry 39, 2399-2405 2000). A single restriction site of Bsu36I was located at the end of LCA sequence. There is no Bsu36I cutting site in the pBN3 vector, which allowed for the use of Bsu36I and Pst I restriction enzymes to cut the pBN3 vector, containing the LCA gene, producing a sticky end for HCA to be ligated. Using forward primer that matches the base pairs of LCA 3′-end with Bsu36I restriction site and reverse primer which is matched with HCA 3′-terminal with built-in 6× His-tag sequence as well as a PstI restriction enzyme site, PCR was performed using genomic DNA as template to creative a HCA gene with N-terminal overhang end of LCA. The PCR product was separated on a 1% DNA agarose gel and purified with a QIAquick® Gel extraction kit (Qiagen, Valencia, Calif.). The purified PCR product was cut with Bsu36I and PstI restriction enzymes, and then ligated to the previously cut pBN3 vector, containing the E224A/E262A BoNT LC gene.

### Example II

### Primer Design and PCR Reaction

To create HCA gene with N-terminal overlap with C-terminal of the LCA both forward and reverse primers were designed as follows. The forward primer with Bsu36I site was: 5′-GGCCGCCCCGGGCGATAAAT ATAGTACCTAAGGTAAATTACAC-3′ (SEQ ID NO:1), and the reverse primer with 6× His-tag and Pst I site was: 5′-AAATTATAATAAACTGCAGG CCTTAGTGATGGTGATGGTGATGCCCGGGAGTTGGCGGGGCCTTCAGTGGCCTTT CTCCCCATCCATCATC-3′ (SEQ ID NO:2). The PCR reactions were performed in 25 μl total volume containing Accuprimer Pfx Supermix, with 200 μM final concentration of each primer, and 300 ng Clostridiumn botulinum type A genomic DNA as template. The PCR sample was preheated at 95° C. for 5 min and then 35 cycles of PCR were performed: 95° C. for 15 seconds, 65° C. for 30 seconds and 68° C. for 2 minutes 48 seconds. After the last cycle, the reaction was incubated for an additional 10 min at 68° C. and 4° for storage.

### Example III

### Cloning HCA Gene into the pBN3 Vector

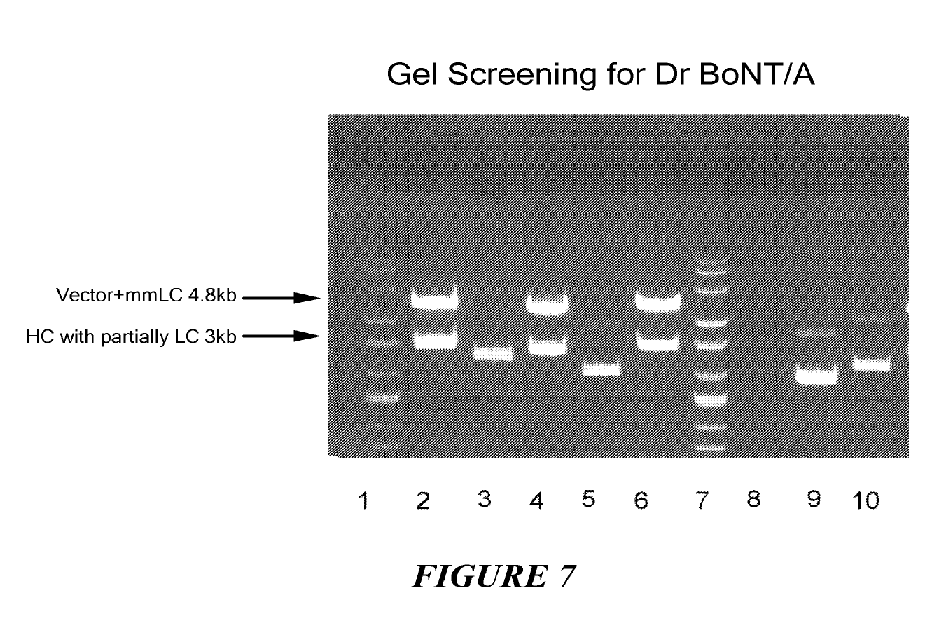

The PCR product was purified using the gel extraction kit (Qiagen, Valencia, Calif.) to remove the excess primers, enzyme, and template and then double digested with Bsu36I and Pst I (New England Bio-Lab, Beverly, Mass.) restriction enzymes in NEB buffer 3. After double digestion, digested products were separated by running-electrophoresis on a low melting point agarose gel and expected band was cut and purified by gel extraction kit. The products were then ligated overnight at 16° C. to the Bsu36I and Pst I digested and dephosphorylated pBN3 vector containing the LCA gene, by using the T4-ligation kits (Novagen, Damstadt, Germany). The ligated reaction mixture was transformed into the E. coli One shoot Top10 competent cells (Invitrogen, Carlsbad, Calif.), and plated on to a LB-agar plate with 100 μm/ml ampicillin for overnight growth. About 100 colonies were obtained from the plate. Several single colonies were picked up to grow overnight in 5 ml LB media with 100 g/ml ampicillin. Plasmid (pBN3-WY3) was isolated with S.N.A.P kit (Invitrogen, Carlsbad, Calif.), and checked with Bsu36I and PstI restriction enzymes. Plasmids with correct DNA size of about 2.8 kb which contain whole HC and partially end of LC were subjected to DNA sequencing. See, FIG. 7.

### Example IV

### Sequencing of the E224A/E262A BoNT/A Gene in the New Construct

The plasmids with correct enzyme cutting pattern harboring the double mutant BoNT/A gene were sent to Genewiz Inc. (North Brunswick, N.J.) for DNA sequencing. Due to the large size of the BoNT/A gene (ca 4 kb), 8 primers were designed as shown below as part of the sequencing strategy.

BoNT/A R518
(SEQ ID NO: 3)
5′-CATAACCATTTCGCGTAAGATTCA-3′
BoNT/A R1190
(SEQ ID NO: 4)
5′-TTGACCATTAAAGTTTGCTGCTA-3′
BoNT/A R1809
(SEQ ID NO: 5)
5′-ACTAATTGTTCTACCCAGCCTAAA-3′
BoNT/A F1649
(SEQ ID NO: 6)
5′-TGTTCCATTATCTTCGTGCTCA-3′
BoNT/A F2103
(SEQ ID NO: 7)
5′-AAGAAATGAAAAATGGGATGAGGT-3′
BoNT/A F2417
(SEQ ID NO: 8)
5′-AACGGTTAGAAGATTTTGATGCT-3′
BoNT/A F2953
(SEQ ID NO: 9)
5′-TGGACTTTACAGGATACTCAGGAA-3′
BoNT/A F3589
(SEQ ID NO: 10)
5′-GCATCACAGGCAGGCGTAGA-3′
(Note: R means reverse primer and F means forward primer)

Eight sets of sequencing data for about 800 bp each were obtained for the mutant BoNT/A gene in the pBN3 vector from plasmid isolated from a single colony. The data were assembled by a computer program (Software-DNASTAR) to produce the full length of the double mutant BoNT/A gene. The designed double mutant, his-tag and the full length BoNT/A gene sequence were confirmed by sequencing data. The new vector harboring the E224A/E262A BoNT/A gene with a his-tag was named pBN3-WY3.

### Example V

### Expression and Purification of the his-Tagged E224A/E262A Double Mutant Recombinant BoNT/A Protein (DR BoNT/A)

The pBN3-WY3 vector was transformed into three different E. coli competent cells: One shot Top 10 competent cells (Invitrogen), BL21(DE3) cell and BL21(DE3) Plys competent cells (Novagen) for pilot expression. These three different competent cells containing pBN3-WY3 expression plasmid were separately grown over night on LB-agar plate with 100 ug/ml ampicillin, followed by 5 ml LB media containing 100 μg/ml ampicillin over night culture. Six liter large scale cultures were grown at 37° C. with Lab-Line Incubator shaker Model 3526 (Melrose, Ill.) at the shaking speed 220 rpm until OD₆₀₀ reached to ˜0.8, 1 mM IPTG was added to induce DR BoNT/A expression. After induction, cell was grown at 25° C. at reduced shaking speed for overnight. The cell cultures were then harvested at 7000 rpm at 4° C. for 10 min.

The cell pellets were resuspended in lysis buffer (50 mM phosphate buffer, pH 8.0, 300 mM NaCl and protein inhibitor cocktail from Roche (Mannheim, Germany) and 0.5 mg/ml lysozyme (Sigma, St Louis, Mo.). The bacteria suspension was incubated on ice for about 30 min and then sonicated to break the cell membrane. After sonication, the lysate was centrifuged by using Sorvall Instruments centrifuge (Model RC5C, SS34 rotor, Belle Mead, N.J.) at 12,000 rpm for about 45 min to remove the insoluble debris. The supernatant obtained from above was loaded to an already equilibrated Ni-NTA column, and the column was washed with the Buffer (50 mM phosphate pH 8.0, 300 mM NaCl, 1 mM PMSF and 20 mM imidazole). DR BoNT/A protein was eluted from the column at 200 mM imidazole in the above buffer. SDS-PAGE (Sodium dodecyl sulfate-polyacrylamide gel electrophoresis) analysis was carried out to check the purity of each fraction at various imidazole concentrations. Fractions with pure DR BoNT/A protein were pooled together and flash frozen in liquid nitrogen, and stored in a −80° C. freezer.

### Example VI

### Protein Concentration and pI Determination

Protein concentrations were determined initially by A280 and A320 readings on a UV-vis spectrophotometer (Jasco, Model 550, Boston Mass.) using a Quartz cuvette of 1 cm path length. The formula that was used to calculate the native toxin concentration: (A280-A320)/1.63× dilute factor (1.63 is the extinction coefficient mg/ml at 280 nm of native toxin). An additional quantitative method using Bio-Rad kits (Hercules, Calif.) with BSA as a standard to obtain protein concentration was also used. Similar results were obtained from both methods.

Isoelectric focusing (IEF) was performed to determine the protein isoelectric point pI using the Phast Gel System (Pharmacia Biotech, Piscataway, N.J.) under IEF conditions. The Phast Gel TM IEF 3-9 (Amersham Bioscience, Sweden) pH range 3-9 was used, and IEF standards pI 4.45-9.6 from Bio-Rad (Hercules, Calif.) were used as markers. Methods for isoelectric focusing involved three steps: a prefocusing step in which the pH gradient is formed of the gel, a sample application step in which sample and marker were applied on the gel, and focusing step in which protein sample is run on the gel and focused at the same point in the gel matching their pI.

Both DR BoNT and native BoNT/A were loaded on to Phast IEF 3-9 gel after dialyzing off the samples to remove chemicals such as urea and ammonium sulfate. The protein concentration of the two proteins used in IEF experiments was 1 mg/ml. After isoelectric focusing, the gel was fixed in 20% TCA (trichloroacetic acid), washed with destaining solution (30% methanol and 10% acetic acid in double distilled (dd) H₂O, and the gel was stained for 1 hour with 0.02% Phast Gel Blue solution prepared in about 30% methanol and 10% acetic acid prepared 0.1% (w/v) CuSO4 solution in ddH2O. The gel was then destained in the destaining solution for several hours. The IEF data from the gel was analyzed by Kodak software of Imagine EL Logic100 machine (Eastman Kodak Company, Rochester, N.Y.). The isoelectric point was also calculated by Proteomics tools from Expasy website based on the amino acid sequence for comparison.

### Example VII

### Determination of Molecular Weight

The molecular mass of the protein was first calculated using Proteomics tools from Expasy® website based on the amino acid sequence. The molecular weight was also determined with SDS-PAGE using Bio-Rad high molecular weight as standards and Imagine EL Logic100 software.

### Example VIII

### Endopeptidase Activity

The endopeptidase activity of DR BoNT was estimated by Enzyme-linked Immunosorbant Assay (ELISA) method established previously (Rigoni et al., (2001) Biochemistry and Biophys, Res. Commun 288, 1231-1237). A 96 well micro-titer plate was used for the assay. 100 μl of 10 μg/ml SNAP-25-GST fusion protein, which is the BoNT/A substrate, was coated in the plate at 37° C. for about 30 min. 200 nM 100 μl each proteins of LCA, E224A/E262A-LCA, native BoNT/A, DR BoNT/A and HCA were added to the plate. After adding protein samples, 1 mM DTT (dithiothreitol) was added in each well. DTT is known to enhance cleavage activity of BoNT/A (Cai et al., (1999) Biochemistry 38, 6903-6910). The cleavage reactions were allowed to incubate at 37° C. for 90 min. The plate was then washed 2 times with PBS buffer pH 7.4, containing 0.1% Tween-20, and then 1 time with PBS without Tween-20. This was followed by addition of 3% BSA dissolved in PBS buffer 100 μl and incubated for 1 h to block the surface. After washing 3 times with PBS buffer, 85 ng/ml Anti-SNAP-25 IgG from rabbit (Stressgen Biotechnologies Corp, Victoria, Canada) was incubated at 37° C. for 1 hour, After washing 3 times with PBS, a peroxidase-labeled anti-rabbit antibody at 1:10,000 dilution from original 0.8 mg/ml was used as the secondary antibody on the plate; the reaction took place at 37° C. for about 1 hour. A substrate solution containing 0.04% OPD (0-phenylenediamine dihydrochloride) and 0.012% H₂O₂ in a 100 mM citrate phosphate buffer, pH 5.0 was used for color development, and the plate was incubated at the room temperature for about 30 min. The reaction was stopped by adding 2 M Sulfuric acid. The color of each well on the plate was measured at 490 nm under a microplate reader (Molecular Devices, Sunnyvale, Calif.). The SNAP-25 protein alone was used as a control, as were BSA, primary antibody, and secondary antibody to determine control amount of SNAP-25 before cleavage, and to ensure background correction.

### Example IX

### Circular Dichroism Spectroscopy

Circular Dichroism (CD) data were collected with a JASCO J-715 spectropolarimeter equipped with a computer-controlled temperature cuvette holder. Far UV CD spectra in the region 180-250 nm were recorded with a 1.0 mm path length cell containing 0.1-0.3 mg/ml protein in 25 mM Tris-HCl, pH 8.0, containing 50 mM NaCl. Typically, a scan rate of 20 nm/min, a response time of 8 second, and a bandwidth of 1.0 nm were used. Spectral resolution was 0.5 nm, and 3 scans were averaged for each spectrum. All spectra were corrected for the signal from buffer. All the far UV CD spectra were recorded at room temperature (25° C.). Mean residue weight ellipticities were used to analyze the CD data for comparison of native BoNT/A with DR BoNT.

### Example X

### Western Blot Assay

For Western blot analysis, 20 μl of 0.57 mg/ml purified DR BoNT/A and 20 μl of 0.53 mg/ml native BoNT/A were loaded in separate wells of the SDS-PAGE gel. The Kaleidoscope prestained standards were used as markers. SDS-PAGE gel of DR BoNT/A and native BoNT/A were transferred to the PVDF membrane using a Bio-Rad semi-dry TRANS-BLOT SD Cell (Hercules, Calif.). The transfer process was carried out at 20 watts for 30 min. The transferred membrane was blocked with 3% BSA in PBS buffer, pH 7.4, for 1 h at room temperature, and then incubated with rabbit anti-BoNT/A antibody 0.5 μg/ml (BBTech, Dartmouth, Mass.) for 1 h at room temperature (25° C.). The membrane was then washed 2× with PBST (PBS with 0.1% Tween-20), and 1× with PBS. Anti-rabbit antibody conjugated to alkaline phosphatase from Sigma (Chemical Co., St. Louis, Mo.) at 1:30,000 dilution was added as the secondary antibody, the membrane was incubated at room temperature for another 1 hour, and the membrane was washed similar to the previous washing procedure. 10 μl liquid substrate containing BCIP (5-bromo-4-chloro-3-indolyl-phosphate) and NBT (Nitroblue tetrazolium) Blue (Sigma) was added for the color development. See, FIG. 1.

### Example XII

### Trypsin Digestion

The protein concentration of both DR BoNT and native BoNT/A was maintained at 0.5-0.6 mg/ml. The ratio between protein and trypsin (Fermentas, Hanover, Md.) was either 250:1 or 50:1 (w/w). Trypsin digestion was carried out in 50 mM Tris buffer, pH 7.6, containing 200 mM NaCl and 5 mM CaCl₂ at room temperature (25° C.) for various periods (5, 10, 30, and 60 min) of incubation. After a given incubation period 20 μl digested mixture was taken out, and 1 mM PMSF was added to stop the reaction before boiling it for 5 min with SDS-PAGE loading buffer (Sharma and Singh (1998) Journal of Natural Toxins 7, No 3 239-253). The samples were then analyzed on a 12% SDS-PAGE gel.

### Example XIII

### Protein Expression, Purification, and Comparison with BoNT/A

The double mutant plasmid DNA with a His₆-tag on the C-terminal end was transferred to three different competent cells: Top10, B121 (DE3) plys, BL21 (DE3). When the yield of different competent cells was compared, it was found that B121 (DE3) gave the best yield of pure protein, ˜8 mg/L. During the purification of protein from strain BL21 (DE3), different fractions were loaded on the SDS-PAGE gel to check the purity. Fractions containing pure protein were pooled. A small contamination of smaller proteins (<50 kDa) was found and a Centriprep YM50 (Millipore, Bedford, Mass.) was used to remove smaller proteins and to concentrate purified protein, or to change buffer. The protein can be stored by adding 20% glycerol in −80° C. Protein precipitate in 0.38 g/ml ammonium sulfate is stable for weeks at 4° C.

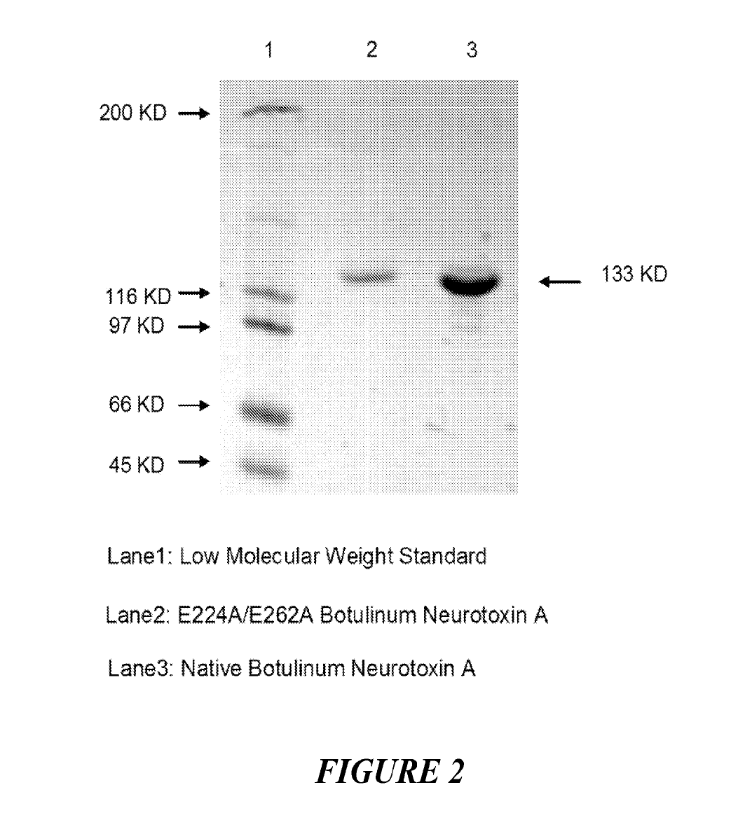

The molecular weight of BoNT/A and DR BoNT/A have been determined with SDS-PAGE gel analysis with Bio-Rad high molecular protein marker as standard. For DR BoNT/A the molecular mass was 132 kDa, and that of the native BoNT/A was 133 kDa. See, FIG. 2. Expasy® software for analysis of protein molecular weight and pI revealed that the molecular weight for BoNT/A and DR BoNT were about 150 kDa, and the pI was 6.

### Example IVX

### Intracellular Delivery of a Drug

This example provides a description of the ability for a drug delivery device as contemplated herein, to provide intracellular delivery of a drug (i.e., for example, a DR BoNT/A related protein, nucleic acid, and/or a small molecule).

A liposome encapsulating DR BoNT/A and/or fragments thereof will be attached to antibodies. The antibodies will have reactivity with a specific diseased tissue (i.e., for example, a cancer tissue). Many cancer specific antigens can be utilized to provide antibodies to allow a targeted delivery of the liposomes. Once the antibodies attach to the cancer specific antigens, the cancer cell engulfs (i.e., for example, by endocytosis) the liposome, wherein the DR BoNT/A related protein, nucleic acid, and/or small molecule is subsequently released into the intracellular space following liposomal dissolution. The released drug will then directly interact with the cancer cell, thereby having a therapeutically beneficial effect.

### Example VX

### DR BoNT/A Binding to SH-SY5Y Cells

This example provides a cell-based assay demonstrating the binding ability of DR BoNT/A.

### Cell Line and Culture Conditions

The human neuroblastoma cell SH-SY5Y was purchased from the American Type Culture Collection (Manassas, Va.). The cells were grown in 1:1 mixture of Eagle's Minimum Essential Medium with non-essential amino acids from ATCC (Manassas, Va.) and Ham's F12 medium from Sigma (St. Louis, Mo.) supplemented with 10% (v/v) fetal bovine serum (ATCC, Manassas, Va.) at 37° C., in a humidified 5% C02 incubator.

### FITC-DR BoNT/A

Recombinant DRBoNT/A was purified in our lab. FITC-labeling of DRBoNT/A was carried out according to the instructions (Sigma).

Treatments of SH-SY5Y Cells with FITC-DRBoNT/A

After seeded the cells in 25 cm² flask for 48 hours, the cells were rinsed with fresh serum free culture medium once, and treated with 40 nM DRBoNT/A in fresh serum free culture medium for 1 hour at 4° C. The cells were washed with PBS and observed by confocal fluorescence microscopy.

### Observations

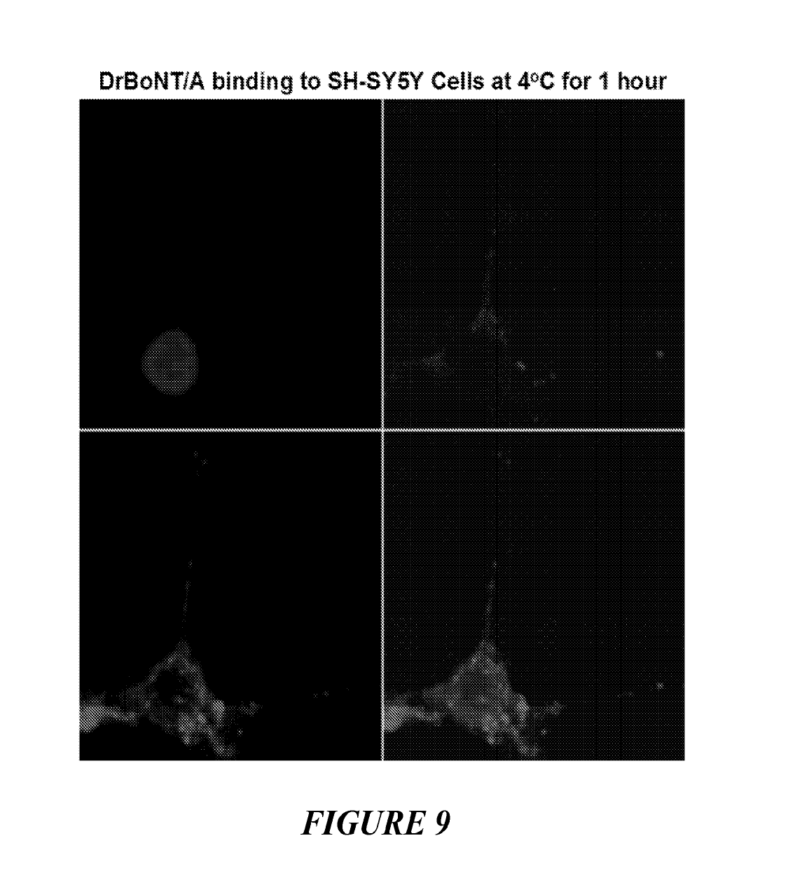

The results show that DRBoNT/A binds to the neuronal cells at the plasma membrane level. See, FIG. 9.

### Example VXI

### Preparation of BoNT/A Mutants

Triple mutants H223M/E224A/E262A was created from a full length BoNT/A. Quadruple mutants H223M/E224A/H227Q/E262A was created from DR BoNT/A. Site specific mutations were generated using the commercially available QuickChange site-direct mutagenesis kit from Stratagene.

MALIH primers:
Forward primer 5′-3′
CCG GCA GTA ACT CTG GCA ATG GCC CTC ATC CAC GCT
GG
Reverse Primer 5′-3′
CCA GCG TGG ATG AGG GCC ATT GCC AGA GTT ACT GCC
GG
MALIQ Primers
Forward primer 5′-3′
CCG GCA GTA ACT CTG GCA ATG GCC CTC ATC CAA GCT
GGTCACCG
Reverse primer 5′-3′
CGG TGA CCA GCT TGG ATG AGG GCC ATT GCC AGA GTT
AC

The PCR reaction for both mutants includes 5 μl reaction buffer, 2 μl plasmid of DR BoNT (8 mg/ml), 1.25 μl each forward and reverse primer (100 ng/ml each primer), 1 ul enzyme and 39.5 μl H₂O, for a total volume of 50 μl. The PCR condition was 95° C. for 30 seconds for hot start, and 17 cycles of 95° C. 30 seconds, 55° C. for 1 minutes and 68° C. for 7 min 30 seconds. After PCR, the PCR product was digested by using DpnI restriction enzyme and then directly transformed to Top10 competent cell, about 100 colonies growing LB agar plate containing 100 μg/ml AMP. Plasmids have been isolated and send to sequence. The sequence results were confirmed the right mutation.

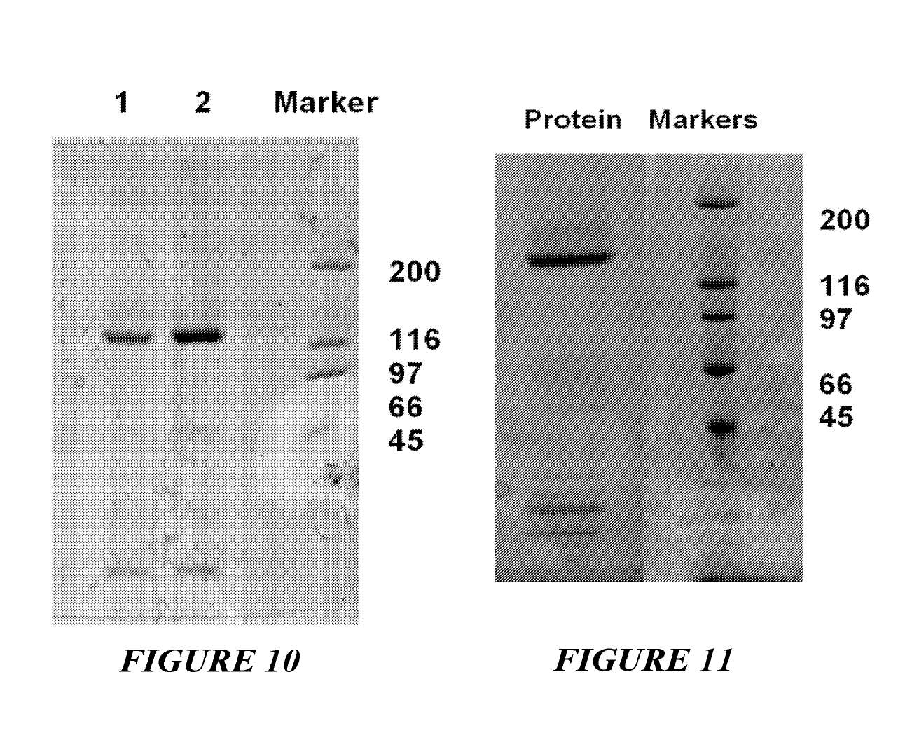

The two mutant plasmids were transformed to BL21 (DE3) competent cell for protein expression. The purification procedure was followed the exact same method as one of DRBoNT/A. After His-tag column chromatography, pure proteins were obtained for both mutants as analyzed by running SDS-PAGE gel. See, FIG. 10 and FIG. 11, respectively.

Primary characterization has been performed for both mutants and neither show no endopeptidase activity (data not shown).

### Example VXII

### Drug Delivery Using a DR BoNT

This example determines the efficacy of delivering a therapeutic compound measured by separation of a drug carrier from a DDV.

3 week old mouse spinal cord neuron cultures were treated for 16 hours with 200 nM fluorescently labeled DDV at 37° C. and then labeled with anti-endosome antibody. Micrographs were obtained on a Bio-Rad 2000 laser confocal microscope using a 100× oil immersion objective. Confocal images were obtained as follows:

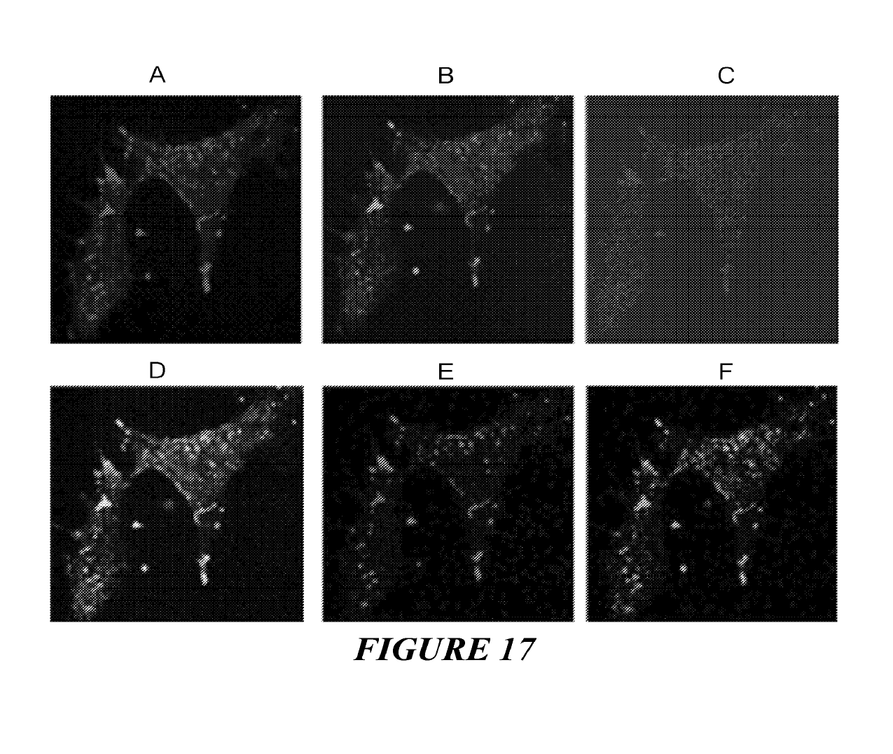

FIG. 17A: red-rHCA: fluorescence elicited at an excitation wavelength of 543 nm;

FIG. 17B: green-OG488-dextran: fluorescence elicited at an excitation wavelength of 488 nm;

FIG. 17C: bright blue-Alexa 633-endosomes: fluorescence elicited at an excitation wavelength of 632 nm;

FIG. 17D: overlay of red and green showing either co-localization (orange) or separation of rHCA and dextran;

FIG. 17E: overlay of red and blue showing either the localization (magenta) of rHCA in the endosomes as believed or its release into the cytosol;

FIG. 17F: overlay of green and blue showing either localization (light blue or greenish blue) of dextran in the endosomes or its release into the cytosol.

The confocal image analysis indicated that about 40% of drug carrier components were separated from DDV and diffused into cytosol from endosome in 3 weeks culture. Results also revealed that the separation of the drug from DDV, as well as neuronal function of glycine release, is cell maturation dependent. These studies suggest that the heavy chain can deliver therapeutic cargo (i.e., for example, an C3E exoenzyme), using a BoNT/A(HC) chimera protein.

### Example VXIII

### DR BoNT Toxicity Assays

This example evaluates the utility of catalytically deactivated BoNT/A (DrBoNT/A) as a drug delivery vehicle.

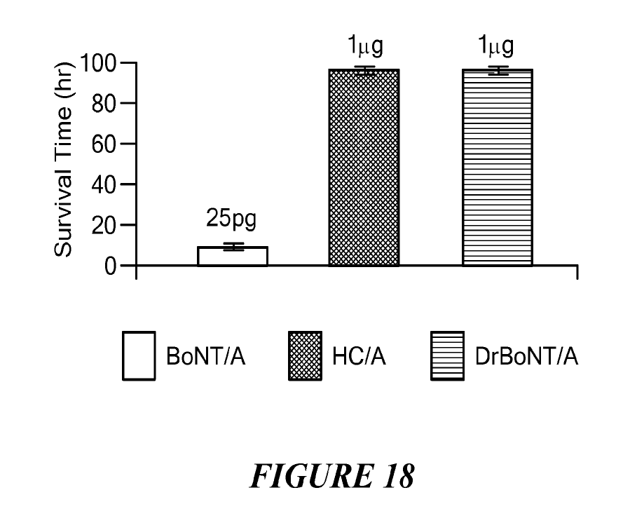

DR BoNT/A in vivo toxicity was determined by mouse bioassay by administering BoNT, HC/A or DR BoNT/A to 6 weeks old Swiss Webster female mice (n=5) via intraperitoneal administration, followed by a 96 h observation period. Mice injected with 25 pg of BoNT/A (1MLD) did not survive more than 10 h, while all the mice survived throughout the observation period even up to 1 μg doses (about 100,000 LD₅₀ dose equivalent of BoNT/A) of DR BoNT/A or HC/A. See, FIG. 18.

These data suggest that catalytically deactivated recombinant BoNT/A Heavy Chain (DR BoNT/A or deactivated HC/A are safe drug delivery vehicles (DDV).

### Example XIX

### Construction and Purification of Chimeric BoNT-C3E Fusion Proteins

This example outlines a proof of concept for a BoNT-based delivery system by preparing BoNT binding and translocation fragment fusion proteins with a C3E exoenzyme and test its functional delivery by assaying the ADP-ribosylation of RhoA in neuronal cells, and to assay neurite outgrowth.

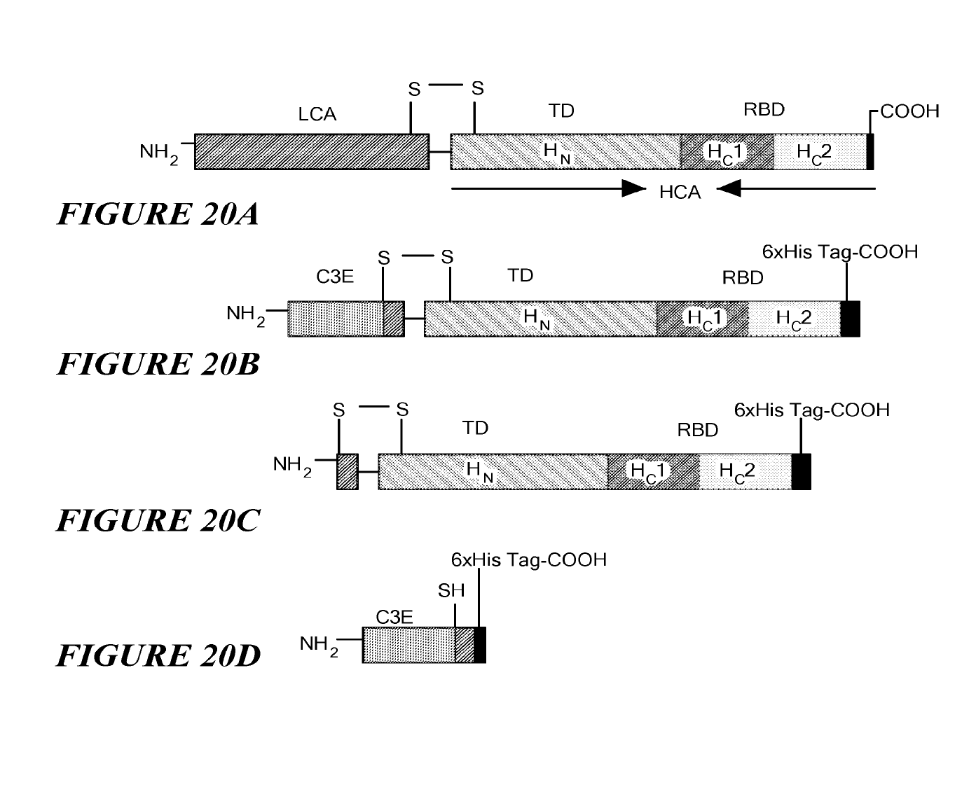

Several ‘test and control’ constructs are engineered by recombinant DNA technology. For example, C3E, (23 kDa) is expressed as Heavy chain A (HC/A, 100 kDa), a fusion protein (e.g. BoNT-C3E, 123 kDa) by standard cloning protocols with C-terminal 6×Histine purification tag. See, FIGS. 20A-20D. The presence of a C-terminal tag adjacent to RBD does not affect the binding and internalization of the holotoxin. An N-terminal amino acid stretch of 25 amino acid residues of light chain A (LCA) will also be included with HC/A and with the BoNT-C3E fusion protein. See, FIGS. 20C and 20B, respectively. Inclusion of this sequence stretch would conserve a nicking region to physically disassociate the LC cargo region by host cell proteases and also provide a cysteine residue (—SH) to aid in the formation of a disulfide bridge with the LC cargo region. Presence of such a disulfide bridge with an BoNT LC cargo region had been previously reported. Maksymowych et al., “Structural features of the botulinum neurotoxin molecule that govern binding and transcytosis across polarized human intestinal epithelial cells” J. Pharmacol. Exp. Ther. 310:633-641 (2004). A similar sequence would be included with C3E to also provide a cysteine residue aiding its fluorescent dye labeling, since C3E do not have cysteine residue in their natural sequence. See, FIG. 20D.

These recombinant proteins would be purified by nickel column affinity chromatography for further studies, and endotoxin free formulations would be tested to elucidate the efficacy of the BoNT-C3E chimera in axonal regenerative therapy using in vivo animal models of SCI.

### Example XX

### Bioactivity Assay

This example describes one assay to determine a recombinant C3E protein and/or BoNT-C3E chimera protein biological activity (e.g., ribosyltransferase activity).

Nicking a BoNT/A-C3E protein with trypsin cleaves at a site in the N-terminal end of the HC/A to dissociate a HC/A region from a LC/A region. Yang et al., “Expression, purification and comparative characterization of deactivated recombinant botulinum neurotoxin type A” The Botulinum J. 1:219-241 (2009). Subsequent reduction of the disulfide linkage is often performed for potency assays and to determine endopeptidase activity of the BoNTs.

Similar reactions would be performed with C3E and/or BoNT-C3E constructs at two different pH conditions (e.g. for example, pH 7.3 or pH 5 that mimics endosomal pH), in order to evaluate the efficiency of C3E dissociation, by analyzing through SDS-Polyacrylamide Gel Electrophoresis (SDS-PAGE). This reaction verifies that the recombinant C3E cargo dissociated as a wild type BoNT LC/A would dissociate.

In vitro ADP-ribosylation reaction, which uses recombinant RhoA as a substrate would be performed with the C3E and/or BoNT-C3E chimera to ensure that the extra tag sequences in the C-terminus of C3E (intended to fuse to the HCA) is not inhibitory to the catalytic activity of C3E upon its release. Aliquots of in vitro ADP-ribosylation reaction which uses recombinant RhoA as substrate would be resolved on SDS-PAGE for western blot detection. Laplante et al., “RhoA/ROCK and Cdc42 regulate cell-cell contact and N-cadherin protein level during neurodetermination of P19 embryonal stem cells” J. Neurobiol. 60:289-307 (2004). Gel mobility shift of ADP-ribosylated RhoA, by the nicked and unnicked B-C3E would be compared by immunodetection using commercially available anti-RhoA specific antibodies, and suitable secondary antibodies.

### Example XXI

### Characterization of Binding, Internalization and Cytoplasmic Localization

For characterizing BoNT-C3E binding and/or internalization human neuroblastoma cell line SH-SY5Y would be used. SH-SY5Y cell lines had been established to study the internalization and activity of botulinum neurotoxin A, and also as cell culture model to study RhoA signaling and neurite outgrowth assays. Purkiss et al., “Clostridium botulinum neurotoxins act with a wide range of potencies on SH-SY5Y human neuroblastoma cells” Neurotoxicol. 22:447-453 (2001); and Wang et al., “Ibuprofen Enhances Recovery from Spinal Cord Injury by Limiting Tissue Loss and Stimulating Axonal Growth” J. Neurotrauma 26:81-95 (2009).

Cellular entry of BoNT-C3E would be characterized by comparing cell binding and internalization of BoNT-C3E with C3E (as control) by fluorescent dye labeling methods, using commercially available kits and by examining under laser scanning fluorescent microscopy. SH-SY5Y cells are routinely grown.

To study cell-surface binding and internalization cells (1×10⁵) plated on coverslips would be incubated with AlexaFluor-488 (green) labeled BoNT-C3E or C3E at 4° C. for up to 30 min in HBSS/BSA, washed with cold HBSS and fixed with 4% paraformaldehyde (PFA) in PBS for 20 minutes. Couesnon et al., “Differential entry of botulinum neurotoxin A into neuronal and intestinal cells” Cell Microbiol. 11:289-308 (2008).

Similarly, for internalization experiments, cells would be incubated with labeled BoNT-C3E or C3E at 37° C. for 5 min, washed with HBSS, and either fixed with paraformaldehyde (PFA) for 15 min or further incubated (for 1 h or 3 h) at 37° C. in HBSS before fixation. After washing with PBS, quenching with 50 mM NH₄Cl in PBS, cells would be permeabilized with 0.2% TritonX-100 in PBS. Cells would be then incubated with wheat germ agglutinin AlexaFluor-594 conjugate (red) and Hoechst 33342 (blue) for 10 min to label plasma membrane or nucleus, respectively.

The cytoplasmic localization of C3E delivery in SH-SY5Y cells would also be assessed using anti-C3E antibodies, followed by using secondary antibodies conjugated with AlexaFluor-488. The Plasma membrane and nuclear labeling will be done as described above. An alternative fluorescein dye labeling method which label proteins through sulfhydryl-reactive groups of cysteine would be done, if needed.

### Example XXII

### RhoA-ADP Ribosylation

After characterizing the cytoplasmic delivery of C3E, the biological activity of blocking RhoA by ADP-ribosylation would be determined in SH-SY5Y cells through gel mobility shift assay examined by western blotting and immunodetection. The cell protein extracts from both SH-SY5Y cells, (post-nuclear extracts) from untreated, BoNT-C3E, C3E or heavy chain (HC/A) treated cells (for 6 h, 12 h and 24 h of incubation time), would be resolved on 12.5% SDS-PAGE for Western blot detection. Laplante et al., “RhoA/ROCK and Cdc42 regulate cell-cell contact and N-cadherin protein level during neurodetermination of P19 embryonal stem cells” J. Neurobiol. 60:289-307 (2004).

As a control, C3E treatment would be included along with the experimental set-up, since they are reported to enter by pinocytosis albeit at lower efficiency. Han et al., “Crystal structure and novel recognition motif of Rho ADP-ribosylating C3 exoenzyme from Clostridium botulinum: structural insights for recognition specificity and catalysis” J. Mol. Biol. 305:95-107 (2001). SH-SY5Y cells treated with HC/A are included as another control, to examine if the heavy chain binding events, which has been reported to trigger neuritogenic properties, is through RhoA-ADP ribosylating mechanisms. Coffield et al., “Neuritogenic actions of botulinum neurotoxin A on cultured motor neurons” J. Pharmacol. Exp. Ther. 330(1):352-358 (2009).

Gel mobility shift of ADP-ribosylated RhoA upon cytoplasmic delivery of C3E would be carried out by immunodetection using commercially available anti-RhoA antibodies; The intracellular ribosylation of RhoA at asn-41 upon treating with BoNT-C3E chimera causes the RhoA immunoreactive band width, anti-RhoA specific antibodies completely shifted towards higher MW by SDS-PAGE. Laplante et al., “RhoA/ROCK and Cdc42 regulate cell-cell contact and N-cadherin protein level during neurodetermination of P19 embryonal stem cells” J. Neurobiol. 60:289-307 (2004).

### Example XXIII

### Neurite Outgrowth Assay

In an effort to assess the axonal regenerative potential of a BoNT-C3E delivery system, a neurite outgrowth assay would be performed using in SH-SY5Y neuroblastoma cells. Wang et al., “Ibuprofen Enhances Recovery from Spinal Cord Injury by Limiting Tissue Loss and Stimulating Axonal Growth” J. Neurotrauma 26:81-95 (2009).

Specifically, a neurite outgrowth assay in the presence of myelin derived inhibitor (MAG) in the growth medium would be performed. MAG diversely inhibits neurite outgrowth by both NgR-dependent and NgR-independent pathways and blockade of RhoA by ADP-ribosylation permits robust neurite outgrowth. So inclusion of MAG in the neurite growth assay is a robust technique to study the effects upon C3E delivery.

A positive control would be, including commercially available RhoA inhibitor, Y-27632 (Calbiochem, USA), that is known to induce neurite out growth in the presence of MAG by blocking ROK (a Rho associated kinase), a downstream target in the RhoA associated signaling pathway. Dergham et al., “Rho signaling pathway targeted to promote spinal cord repair” J. Neurosci. 22:6570-6577 (2002).

The SH-SY5Y cells would be pre-differentiated for 6 days with 2% fetal bovine serum and 10 mM retinoic acid, and added with MAG or to the MAG-expressing CHO cells (which would be plated at 30,000 cells/well, 24-36 h prior to adding pre-differentiated SH-SY5Y neuroblastoma cells). SH-SY5Y Cells would be the grown at 37° C. for 24 h and would be treated with 1-10 μg/ml B-C3E, and HCA, or Y-27632 for another 24 h along with untreated control. After 24 h or 48 h outgrowth period, the cells would be fixed and stained with anti-βIII tubulin antibody. Measurements to estimate neurite outgrowth would be by examining neurites/cell, branch points/neurite, average length of neurites, and percentage of neurite-bearing cells, with neurite defined as a process longer than the cell body.

### Example XXIV

### In Vivo Assessment of BoNT-C3E Efficacy Using SCI Mice Models

This example describes one approach to determine the potency of botulinum mediated C3E delivery on neurite outgrowth by targeting RhoA, and validate the proof of principle in relevant and appropriate animal models by observing behavioral recovery monitoring forelimb-hindlimb movements and coordination.

Objectives are tested and evaluated for therapeutic efficacy of BoNT-C3E chimera proteins by a topical application of BoNT-C3E chimera directly on the lesion site. Dergham et al., “Rho signaling pathway targeted to promote spinal cord repair” J Neurosci. 22:6570-6577 (2002). BALB-c female mice (n=70) weighing approximately 20 gm would be anesthetized with 0.4 ml/kg hypnorm and 5 mg/kg diazepam or by using inhalation of 4.0% isoflurane in N2O/O2 (70:30).

A segment of the thoracic spinal cord would then be exposed using fine rongeurs to remove the bone and a dorsal over-hemisection would be made at T7. Dorsal part of the spinal cord would be cut using fine scissors, which would be cut second time with a fine knife to ensure that the lesion extends past the central canal. A fibrin adhesive delivery system based on aprotinin, thrombin and fibrinogen (Tisseel V H KIT, ImmunoAG) would be prepared as per manufacturer instructions and reconstituted with B-C3E or C3E protein mixture. The formulation would be applied to the spinal cord. As controls, second group of mice would be treated with similar fibrin adhesive formulation infused with buffer but without B-C3E or C3E and a third group would be left untreated.

### Example XXV

### Behavioral Recovery Analysis

Mice would be given behavioral examinations preoperatively and at 2 d and 1, 2, and 4 weeks after neuronal surgery and would be assessed according to a modified Basso-Beattie-Bresnahan (BBB) locomotor rating scale with 17 point score to analyze individual components of limb movement, weight support, plantar and dorsal stepping, forelimb-hindlimb coordination, paw rotation, toe clearance, trunk stability, and tail placement. Basso et al., “A sensitive and reliable locomotor rating scale for open filed testing in rats” J. Neurotrauma 12:1-21 (1995); and Dergham et al., “Rho signaling pathway targeted to promote spinal cord repair” J. Neurosci. 22:6570-6577 (2002).

For scoring, each animal would be videotaped for 3 min. In the late phase of recovery, the BBB score was determined from sequences of four steps or more from digitized videos projected on a computer screen at one-fourth speed. Detailed patterns of front paw and foot placements would be assessed.

Further studies would evaluate the retrograde and transneuronal delivery potential of BoNT/A-C3E chimera, by administering through intramuscular injection route in appropriate SCI injury models.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A composition comprising a chimeric protein comprising a C3E-exoenzyme protein and a non-toxic neurotoxin heavy chain.

2. The composition of claim 1, wherein said neurotoxin heavy chain comprises a Clostridium botulinum neurotoxin heavy chain.

3. The composition of claim 1, wherein said chimeric protein further comprises neurotoxin light chain.

4. The composition of claim 3, wherein said neurotoxin light chain lacks an endopeptidase region.

5. The composition of claim 3, wherein said neurotoxin light chain comprises at least one mutation.

6. The composition of claim 3, wherein said neurotoxin light chain comprises at least two mutations.

7. The composition of claim 5, wherein said at least one mutation is selected from at least one of the group consisting of H223M, H227Q, E224A or E262A.

8. The composition of claim 1, wherein said C3E-exoenzyme protein is a Clostridium botulinum C3E-exoenzyme protein.

9. The composition of claim 1, wherein said C3E protein and said heavy chain are linked by a disulfide bridge.

10. The composition of claim 1, wherein said C3E protein is attached to the light chain.

11. A method, comprising:
a) providing;
i) a patient having a nerve tissue injury;
ii) a composition comprising a chimeric protein comprising a C3E protein and a non-toxic neurotoxin heavy chain;
b) administering said composition to said patient wherein said nerve injury is at least partially regenerated.

12. The method of claim 11, wherein said nerve tissue injury comprises a spinal cord injury.

13. The method of claim 11, wherein said administering is selected from at least one of the group consisting of topical, intramuscular, intraspinal and intrathecal.

14. The method of claim 11, wherein said neurotoxin heavy chain comprises a Clostridium botulinum neurotoxin heavy chain.

15. The method of claim 11, wherein said chimeric protein further comprises neurotoxin light chain.

16. The method of claim 15, wherein said neurotoxin light chain lacks an endopeptidase region.

17. The method of claim 15, wherein said neurotoxin light chain comprises at least one mutation.

18. The method of claim 15, wherein said neurotoxin light chain comprises at least two mutations.

19. The method of claim 17, wherein said at least one mutation is selected from at least one of the group consisting of H223M, H227Q, E224A and E262A.

20. The method of claim 11, wherein said C3E-exoenzyme protein is a Clostridium botulinum C3E-exoenzyme protein.

21. The method of claim 11, wherein said C3E protein and the heavy chain are linked by a disulfide bridge.

22. The method of claim 15, wherein said C3E protein is attached to said neurotoxin light chain.
